(19) United States
(12) Patent Application Publication
(10) Pub. No.: US 2004/0063902 A1
(43) Pub. Date: Apr. 1, 2004

(54) Side chain anchored thioester and selenoester generators

(76) Inventor: Leslie Philip Miranda, San Francisco, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/622,359

(22) Filed: Jul. 18, 2003

Related U.S. Application Data
(60) Provisional application No. 60/398,891, filed on Jul. 25, 2002.

(51) Int. Cl. C12N 9/16

(52) U.S. Cl. 530/324; 435/196; 530/333

(57) ABSTRACT

Thioester and selenoester generators, thioester and selenoester compounds, and related methods for their production are provided. The subject thioester and selenoester generators include an amino acid synthon having an N-terminal group joined to a C-terminal group through an organic backbone comprising one or more carbons. The organic backbone contains a carbon having a side chain anchored to a support through a nucleophile-stable linker and is lacking reactive functional groups. The organic backbone may include a target molecule of interest, such as an amino acid, peptide, polypeptide or other organic compound of interest, and/or the N- and/or C-termini can be elaborated using a variety of synthesis approaches to provide a target molecule of interest. The compounds and methods find a wide variety of uses, including use in thioester- or selenoester-based chemical ligation techniques.

### CROSS-REFERENCE TO RELATED APPLICATION

This application claims the benefit of U.S. provisional application serial no. 60/398,891, filed Jul. 25, 2002, which application is incorporated herein by reference in its entirety.

### BACKGROUND OF THE INVENTION

Thioesters and selenoesters represent an important class of molecules that readily react with nucleophiles. Thioesters are particularly useful for conjugation and chemoselective ligation reactions. Chemical ligation involves the chemoselective covalent linkage of a first chemical component to a second chemical component. Unique, mutually reactive functional groups present on the first and second components can be used to render the ligation reaction chemoselective. For example, thioesters are commonly used to direct the chemoselective chemical ligation of peptides and polypeptides. Several different thioester-mediated chemistries have been utilized for this purpose, such as native chemical ligation (Dawson, et al., Science (1994) 266:776-779; Kent, et al., WO 96/34878; Kent, et al., WO 98/28434).

Unfortunately, conventional preparation and use of peptide and other thioesters (Hojo, et al. Pept. Chem. (1992), Volume Date 1991, 29th pp.115-20; Canne, et al. Tetrahed. Letters (1995) 36:1217-20; Hackeng, et al. Proc Natl Acad Sci USA. (1999) 96:10068-73) have been limited to non-nucleophilic synthetic strategies. For example, when attempting to make thioester-activated peptides using N-α-9-fluorenylmethyloxycarbonyl (“Fmoc”)-based synthesis, the unwanted destruction of the thioester moiety by nucleophiles such as piperidine or piperidine-generated hydroxide ions during synthesis of the peptide will occur. This is a significant problem, since the preferred reagent employed to remove Nα-Fmoc groups in each cycle of Fmoc-based organic synthesis contains piperidine. Piperidine, like other strongly basic or nucleophilic compounds (hereinafter “nucleophiles,”) destroys the thioester component of the peptide, rendering it useless for subsequent thioester-mediated reactions.

Several attempts have been made to address this problem. In one of the more promising approaches, Botti et al. (WO 02/18417) have reported on the application of nucleophile-stable carboxyester thiols or orthothioloester compounds for generating thioester and selenoester compounds. However, other efforts have met with limited success. For instance, Clippingdale et al. (J. Peptide Sci. (2000) 6:225-234) have used a non-nucleophilic base to remove Nα-Fmoc groups of peptides made using Fmoc-based Solid Phase Peptide Synthesis (“SPPS”). This method has several problems, including generation of unwanted deletions, side-products, and requirement for backbone protection strategies. Other groups, including, Bertozzi et al. (J. Amer. Chem. Soc. (1999) 121:11684-11689) and Pessi et al. (Journal of the American Chemical Society; 1999; 121:11369-11374.), have reported adapting Fmoc SPPS in combination with a ‘Kenner’ safety-catch linker, which is stable to nucleophiles until the linker has been alkylated, to produce a fully protected peptide-thioester in solution. A drawback of this technique is the poor solubility properties of protected peptides in solution, as well as side reactions inherent to the method, such as the formation of unwanted alkylated byproducts when the linker is alkylated to render it labile, and thus it is impractical for many applications.

In addition, Barany et al. (J. Org. Chem. (1999) 64(24):8761-8769) have reported on a Fmoc-SPPS method employing a backbone amide linker (“BAL”) to generate peptide thioesters on-resin. Among other problems, the BAL method is prone to diketopiperazine formation in the first few peptide extension cycles, reducing yields and its general application. Ishi et al. (Biosci. Biotechnol. Biochem. (2002) 66(2):225-232) have reported on the use of Fmoc-SPPS to generate Fmoc protected glycopeptide thioesters. As noted above, removal of Fmoc protecting groups is incompatible with thioesters, limiting the utility of this approach. Moreover, beyond a requirement for a serine or threonine anchored to a silyl ether linker based resin, the Ishi et al. method generates thioester products that are fully or substantially protected when released from the resin into solution. As noted above, such protected products exhibit poor solubility in solution, particularly in aqueous-based solutions. Similar frustration has been experienced in nucleophilic-based synthesis schemes for molecules other than peptides, such as small organic molecules.

Accordingly, there is a need for a universal and robust system for producing thioester- and selenoester-generating compositions compatible with organic or aqueous reaction conditions for use in various organic synthesis strategies, and conjugation and chemoselective ligation reactions that employ thioester- or selenoester-mediated reactions. The present invention satisfies these needs, as well as others, and generally overcomes deficiencies found in the background art.

### SUMMARY OF THE INVENTION

The invention provides thioester- and selenoester-generators, thioester and selenoester compounds, and related methods for their generation. The thioester and selenoester generators of the invention, in one embodiment, comprise an amino acid synthon having an N-terminal group joined to a C-terminal group through an organic backbone comprising one or more carbons, where the organic backbone comprises a carbon having a side chain anchored to a support through a nucleophile-stable linker and is lacking reactive functional groups, and where (i) the N-terminal group comprises an unprotected or protected N-terminal group, with the proviso that the N-terminal protecting group is removable under non-nucleophilic conditions, and the C-terminal group comprises a moiety selected from the group consisting of a thioester or selenoester, or where (ii) the N-terminal group comprises an unprotected or protected N-terminal group, and the C-terminal group comprises a moiety selected from the group consisting of a sterically hindered thioester or selenoester.

The invention also provides methods for production of thioester and selenoester generators. In one embodiment, a method is provided for the production of a sterically hindered or non-hindered thioester and selenoester generators comprising:

(a) providing a composition comprising an amino acid synthon having an N-terminal group joined to a C-terminal group through an organic backbone comprising one or more carbons, where the organic backbone comprises a carbon having a side chain anchored to a support through a nucleophile-stable linker and is lacking reactive functional groups, and where (i) the N-terminal group comprises an unprotected or protected N-terminal group, with the proviso that the N-terminal protecting group is removable under non-nucleophilic conditions, and the C-terminal group comprises a free carboxyl, or (ii) the N-terminal group comprises an unprotected or protected N-terminal group, and the C-terminal group comprises a free carboxyl; and

(b) converting the free carboxyl of the product of step (a)(i) to a thioester or selenoester, or of step (a)(ii) to a sterically hindered thioester or sterically hindered selenoester.

In another embodiment, a method is provided for the nucleophile-based production of sterically hindered or non-hindered thioester and selenoester generators comprising:

(a) providing a composition comprising an amino acid synthon having an N-terminal group joined to a C-terminal group through an organic backbone comprising one or more carbons, where the N-terminal group comprises a reactive functional group protected with a nucleophile-labile protecting group, the C-terminal group comprises a carboxyl protected with a carboxyl protecting group which is removable under conditions orthogonal to the nucleophile-labile protecting group, and the organic backbone is lacking reactive functional groups and comprises a carbon having a side chain anchored to a support through a nucleophile-stable linker cleavable under conditions which are orthogonal to the carboxyl protecting group;

(b) removing the nucleophile-labile protecting group from the composition of step (a) under nucleophilic conditions and forming an N-terminal group comprising a first reactive functional group;

(c) coupling to the product of step (b) a compound that forms a covalent bond with the first reactive functional group to form an elongated product, where the compound is selected from the group consisting of: (i) an unprotected compound comprising a single reactive moiety that forms the covalent bond with the first reactive functional group; (ii) a protected compound comprising a single reactive moiety that forms the covalent bond with the first reactive functional group, and an amine protected with a nucleophile-stable amino protecting group that is removable under conditions orthogonal to the removal of the carboxyl protecting group; and (iii) a protected compound comprising a single reactive moiety that forms the covalent bond with the first reactive functional group, and one or more additional reactive functional groups protected with a protecting group that is removable under conditions orthogonal to the removal of the carboxyl protecting group;

(d) removing from the product of step (c) the carboxyl protecting group to generate a free carboxyl group; and

(e) converting the free carboxyl group of the product of step (d) to a thioester or selenoester, with the proviso that the converting the product of step (d) formed from the elongated product of step (c)(iii) comprises generating a sterically hindered thioester or selenoester.

The invention also provides methods for the generation or synthesis of sterically hindered or non-hindered thioester and selenoester compounds. The methods include, providing a thioester or selenoester generator of the invention and cleaving the linker under non-nucleophilic conditions so as to generate a thioester or selenoester compound free of the support. Thioester or selenoester compounds produced in accordance with this method comprise an amino acid synthon having an N-terminal group joined to a C-terminal group through an organic backbone comprising one or more carbons, where (i) the N-terminal group comprises an unprotected or protected N-terminal group, with the proviso that the N-terminal protecting group is removable under non-nucleophilic conditions, and the C-terminal group comprises a moiety selected from the group consisting of a thioester or selenoester, or (ii) the N-terminal group comprises an unprotected or protected N-terminal group, and the C-terminal group comprises a moiety selected from the group consisting of a sterically hindered thioester or selenoester.

The thioester and selenoester generating compounds, the resulting thioester and selenoester compounds themselves, and the related methods greatly expand the capabilities of solid phase synthesis schemes that employ or benefit from the use of thioesters or selenoesters, particularly for synthesis of target molecules by nucleophilic schemes such solid phase Fmoc-based peptide synthesis. The invention allows for the introduction of a variety of thioester and selenoester functionalities onto a target molecule of interest, particularly peptides and polypeptides. The invention may be employed in a wide range of thioester and selenoester mediated ligation reactions for production of peptides, polypeptides and other organic molecules capable of being constructed using ligation schemes employing thioesters and/or selenoesters. These and other objects and advantages of the invention will be apparent from the detailed description below.

### BRIEF DESCRIPTION OF THE DRAWINGS

The invention will be more fully understood by reference to the following drawings, which are for illustrative purposes only.

FIG. 1 is a reaction scheme illustrating an overview of the synthesis of thioester and selenoester generators and thioester and selenoester peptides in accordance with the invention.

FIG. 2 is a reaction scheme illustrating the synthesis of a peptide thioester in accordance with the invention using a side-chain anchored glutamic acid for Fmoc/SPPS.

FIG. 3 is a reaction scheme illustrating the synthesis of a peptide thioester in accordance with the invention using a side-chain anchored lysine for Fmoc/SPPS.

### DETAILED DESCRIPTION OF THE INVENTION

Disclosed herein are thioester- and selenoester-generators, thioester and selenoester compounds, and related methods for thioester generation. The compounds and methods have wide applicability in organic synthesis for the generation of activated thioester and selenoesters. The subject compounds are particularly useful in peptide and polypeptide synthesis techniques that employ thioester and/or selenoester-mediated ligation, including native chemical ligation. The invention allows generation of activated thioesters and selenoesters from precursors that are prepared under strong nucleophilic conditions such as those occurring in Fmoc- (Nα-9-fluorenylmethyloxycarbonyl)-based peptide synthesis. The compounds of the invention support complex multi-step ligation or conjugation schemes.

The invention is described primarily in terms of use with Fmoc-compatible synthesis, including Fmoc-based solid-phase peptide and polypeptide synthesis (SPPS). Those skilled in the art will recognize, however, that the invention may be used for preparation of a variety of compounds having nucleophile-sensitive functionalities using various nucleophile-labile protecting group schemes. Moreover, those skilled in the art will recognize that the thioester and selenoester generators and related methods of the invention may be applied in tert-butyloxycarbonyl- (Boc) compatible synthesis, including Boc-based SPPS, as well as combinations of Fmoc- and Boc-compatible synthesis. Additional embodiments include 2-(4-nitrophenylsulfonyl)ethoxycarbonyl (Nsc .Bzi), allyloxycarbonyl (Alloc), and other protection schemes compatible with SSPS. The invention is also described primarily in terms of peptide synthesis involving chain extension from an Nα terminus. Those skilled in the art will recognize that peptide synthesis involving extension from the C-terminus may also be carried out using the invention. Thus, it should be understood that the invention is not limited to the particular embodiments described below, as variations of these embodiments may be made and still fall within the scope of the appended claims. It should also be understood that the terminology employed is for the purpose of describing particular embodiments, and is not intended to be limiting. Instead, the scope of the present invention will be established by the appended claims. Any definitions herein are provided for reason of clarity, and should not be considered as limiting. The technical and scientific terms used herein are intended to have the same meaning as commonly understood by one of ordinary skill in the art to which the invention pertains.

### Thioester and Selenoester Generators

The thioester and selenoester generators of the invention include, in general terms, an amino acid synthon having an N-terminal group joined to a C-terminal group through an organic backbone comprising one or more carbons. The organic backbone comprises a carbon having a side chain anchored to a support through a nucleophile-stable linker, and is lacking reactive functional groups. The organic backbone may comprise a target molecule of interest, such as an amino acid, peptide, polypeptide or other organic compound of interest, and/or the N- and/or C-termini can be elaborated using a variety of synthesis approaches to comprise a target molecule of interest. The linker may also comprise a variety of linkers cleavable under non-nucleophilic conditions, such as linkers cleaved by strong acid, reduction, displacement reagents, light, and the like, and may include a target molecule of interest, or components of a target molecule, and can be of variable lengths.

In certain embodiments, the thioester- or selenoester-generators of the invention bear an N-terminal group having a moiety selected from: (i) a functional group protected with a nucleophile-labile protecting group, (ii) a functional group protected with a nucleophile-stable protecting group, (iii) an unprotected functional group, or (iv) an unprotected group that is substantially unreactive under conditions employed for generating the thioester- and selenoester-generators of the invention. A preferred N-terminal group comprises a moiety selected from a free amine, an amine protected with a nucleophile-stable amine protecting group, and an unprotected group lacking a reactive functionality, such as a unreactive alkyl or aryl capping moiety that may be linear, branched, substituted or unsubstituted.

In certain embodiments, the thioester- or selenoester-generators of the invention possess a C-terminal group having a moiety selected from: (i) a carboxyl protected with a carboxyl protecting group removable under conditions orthogonal to the N-terminal nucleophile-stable protecting group and linker, or (ii) a thioester or selenoester. The thioester- or selenoester-generators may comprise sterically hindered or non-hindered thioester or selenoester.

The thioester- or selenoester-generators of the invention may be provided with such N- and C-terminal groups in various combinations, depending on the intended end use. As described above, the thioester and selenoester generators comprise an amino acid synthon having an N-terminal group joined to a C-terminal group through an organic backbone comprising one or more carbons, where the organic backbone comprises a carbon having a side chain anchored to a support through a nucleophile-stable linker and is lacking reactive functional groups. In a preferred embodiment, the N-terminal group comprises an unprotected or protected N-terminal group, with the proviso that the N-terminal protecting group is removable under non-nucleophilic conditions, and the C-terminal group comprises a moiety selected from the group consisting of a thioester or selenoester. In another preferred embodiment, the N-terminal group comprises an unprotected or protected N-terminal group, and the C-terminal group comprises a moiety selected from the group consisting of a sterically hindered thioester or sterically hindered selenoester.

By “amino acid synthon” is intended a structural unit within a molecule, the structural unit comprising an amino acid or amino acid residue having an N-terminus comprising or extending from the alpha nitrogen of the amino acid or amino acid residue, a C-terminus comprising or extending from the alpha carbonyl of the amino acid or amino acid residue, and an organic backbone that joins the N- and C-termini and is substituted or unsubstituted with one or more side chains, where the structural unit can be formed and/or assembled by known or conceivable synthetic operations.

Examples of amino acid synthons are unprotected and partially or fully protected amino acids and peptides having a modified or unmodified alpha amino terminus (N-terminus) and/or a modified or unmodified alpha carbonyl terminus (C-terminus). This includes unactivated and activated esters thereof, as well as salts thereof, such as trifluoroacetic acid (TFA) salts. It also includes variable forms thereof in which the pendant N- and/or C-termini comprise terminal groups other than an alpha amino or carbonyl moiety, such as other amino acid non-functional and functional groups, one or more protecting groups, halogens, azides, conjugates, organic moieties other than an amino acid, a target molecule of interest, or components thereof, depending on the intended end use.

The term “amino acid” means any of the 20 genetically encodable amino acids, non-encoded amino acids, and analogs and derivatives thereof, including α-amino acids, β-amino acids, γ-amino acids, and other compounds having at least one N-terminal amino functionality and at least one C-terminal carboxyl (or carbonyl) functionality thereon. L- and D-forms of the chiral amino acids are also contemplated. The terms “peptide”, “polypeptide,” and “protein”, which may be used interchangeably herein, refer to an oligomeric or polymeric form of amino acids, which can include coded and non-coded amino acids, chemically or biochemically modified or derivatized amino acids, and polypeptides having modified peptide backbones.

In the context of an amino acid synthon, an “organic backbone” may comprise the alpha, beta and/or gamma carbons of a single amino acid residue, and other substituents, including additional backbone carbons and/or heteroatoms, as well as alpha amino groups of an amino acid or residue that are substituted or unsubstituted (amides included), alpha carbonyls that are substituted or unsubstituted (carboxyls, carboxyesters, and amide bonds included), and may comprise an amino acid residue or peptide, as well as organic side chains. Representative organic side chains are those of amino acids. The organic backbone typically comprises part or most of a target molecule of interest.

By “lacking reactive functional groups” is intended a group or radical in which such reactive functional groups are entirely absent, as well as a group or radical that contain protected functional groups that would otherwise be reactive but for the presence of the protecting group(s).

Accordingly, the organic backbone may be fully protected, partially protected or unprotected depending on the intended end use. For example, the organic backbone may have one or more side chains bearing a functional group protected with a protecting group removable under conditions orthogonal to the N-terminal protecting group. This is particularly convenient where the organic backbone is constructed using Fmoc-compatible synthesis, and the N-terminal protecting group, if present, is removable under conditions orthogonal to Fmoc-removal. In this situation, the organic backbone may include a peptide chain containing amino acid residues bearing protected functional groups removable under conditions orthogonal to nucleophilic removal of an N-terminal Fmoc group during peptide elongation cycles, such as nucleophile-stable/acid-cleavable protecting groups, and where the last amino acid coupling includes an N-terminal protecting group cleavable under conditions different from Fmoc or side-chain protecting group removal, such as catalytic hydrogenation conditions (e.g., an Alloc group).

In other instances, the organic backbone may contain one or more side chains bearing a functional group protected with a protecting group that is removable under the same conditions as the N-terminal protecting group. For example, both the N-terminal protecting group and the side chains can be protected with nucleophile-stable, acid-cleavable protecting groups, so that the side chains and N-terminal group can be deprotected in one step. Particularly useful nucleophile-stable protecting groups cleavable under acidic conditions include the tert-butyl (tBu), tert-butyloxycarbonyl (Boc), and trityl (Trt) groups.

Alternatively, the organic backbone may contain one or more side chain functional groups that are substantially non-reactive to conditions used for generating or manipulating a target molecule attached to the support, and/or side chains that would otherwise be reactive but are protected with protecting groups that are orthogonal to such generating or manipulating conditions.

The term “orthogonal” as used herein with respect to protecting groups, linkers, and other groups means that the specific group or linker is removable or cleavable under conditions that do not result in removal or cleavage of an “orthogonal” group or linker. Thus, for example, where the linker is nucleophile-stable and the N-terminal group bears a nucleophile-labile protecting group, cleavage of the linker is “orthogonal” to removal of the nucleophile-labile protecting group, and vice versa.

For instance, when the organic backbone is made to contain cysteine amino acid residues, the side chain thiol can be protected with an acetamidomethyl (Acm) or Picolyl group, which are stable to basic conditions (e.g., typical conditions for Fmoc-compatible cycles during primary target synthesis) or acidic conditions (e.g., typical Boc-compatible cycles and/or conditions for final deprotection and cleavage of an elongated thioester or selenoester target molecule from the support). Protecting groups like Acm- and Picolyl also are removable under conditions orthogonal to carbonyl protecting groups such as Allyl or ODMab. The same orthogonal protection strategy can be employed with other side chains, for example, side chains bearing a primary amine protected with an Alloc group. Where the organic backbone contains side chain functional groups that are substantially unreactive, protection of those groups is typically not required. Examples of side chain groups that are substantially unreactive include alcohols, and other such groups can be selected depending on the conditions employed.

The above stratagems also can be exploited with respect to the nucleophile-stable linker. For instance, the N-terminal protecting group and the nucleophile-stable linker can be provided in a combination where they are cleavable under orthogonal conditions. Alternatively, the N-terminal protecting group and the nucleophile-stable linker can be selected so that they are both cleavable under the same conditions. Many such linkers are known, and can be selected for this purpose, including those described in further detail herein. Preferred linkers stable to nucleophiles such as piperidine are cleavable under conditions such as acid or light. These include a wide range of linkers, with the most preferred linkers being compatible with Fmoc-based, Boc-based, Alloc-based, and/or peptide synthesis. The linkers may employ multi-detachable components, including dual linker systems, as well as contain spacers or other divalent linker elements.

Linkers usable with the invention include, for example, PAL (5-(4′-aminomethyl-3′,5′-dimethoxyphenoxy)valeric acid, XAL (5-(9-aminoxanthen-2-oxy)valeric acid), 4-(alpha-aminobenzyl)phenoxyacetic acid, 4-(alpha-amino-4′-methoxybenzyl)phenoxybutyric acid, p-alkoxybenzyl (PAB) linkers, photolabile o-nitrobenzyl ester linkers, 4-(alpha-amino-4′-methoxybenzyl)-2-methylphenoxyacetic acid, 2-hydroxyethylsulfonylacetic acid, 2-(4-carboxyphenylsulfonyl)ethanol, (5-(4′-aminomethyl-3′,5′-dimethoxyphenoxy)valeric acid) linkers, WANG hydroxymethyl phenoxy-based linkers, RINK trialkoxybenzydrol and trialkoxybenzhydramine linkers, and Sieber aminoxanthenyl linkers. PAM, SCAL, and other linker systems may also be used. These linker systems are cleavable under well known acidolysis conditions (typically trifluoroacetic acid (TFA) or hydrogen fluoride (HF)), UV photolysis (λ≈350 nm) conditions, or catalytic hydrogenation conditions. Several of the above linker systems are commercially available as pre-formed on resin and glass supports.

The support of the thioester and selenoester generators of the invention comprises a solid phase, matrix or surface compatible with organic synthesis strategies. Preferred supports are those compatible with peptide synthesis. A variety of such supports are well known, and can be employed, including those described in further detail herein. Examples include supports or resins comprising cross-linked polymers, such as divinylbezene cross-linked polystyrene polymers, or other organic polymers that find use for solid-phase organic or peptide synthesis. Controlled porous glass (CPG) supports are another example. In general, the most preferred supports are stable and possess good swelling characteristics in many organic solvents.

With respect to the side chain of the organic backbone that is anchored through the linker to a support, the side chain is preferably an amino acid side chain. Examples of preferred amino acid side chains include those of aspartic acid, glutamic acid, glutamine, lysine, serine, threonine, arginine, cysteine, histidine, tryptophan, tyrosine, and asparagine. These amino acid side chains are particularly useful for traceless cleavage reactions, i.e., reactions where cleavage of the linker regenerates the original side chain, and thus generation of a thioester or selenoester compound bearing no residual linker. Where other amino acid side chains are employed for anchoring to the support, residual linker may be present following cleavage of the linker.

As described above, the thioester and selenoester generators of the invention may have a modified or unmodified alpha amino terminus (N-terminus) and/or a modified or unmodified alpha carbonyl terminus (C-terminus). In a preferred embodiment, the thioester and selenoester generators of the invention have an N-terminal group that comprises an amino acid. Any amino acid can be used. In a preferred embodiment, the amino acid is capable of chemical ligation. Chemical ligation involves the selective covalent linkage of a first chemical component to a second chemical component. Orthogonally reactive functional groups present on the first and second components can be used to render the ligation reaction chemoselective. For example, chemical ligation of peptides and polypeptides involves the chemoselective reaction of peptide or polypeptide segments bearing compatible, mutually reactive C-terminal and N-terminal amino acids. Several different chemistries have been utilized for this purpose, examples of which include native chemical ligation (Dawson, et al., Science (1994) 266:776-779; Kent, et al., WO 96/34878; Kent et al., U.S. Pat. No. 6,184,344), extended general chemical ligation (Kent, et al., WO 98/28434; and Kent et al., U.S. Pat. No. 6,307,018); extended native chemical ligation (Botti et al., WO 02/20557); oxime-forming chemical ligation (Rose, et al., J. Amer. Chem. Soc. (1994) 116:30-33), thioester forming ligation (Schnölzer, et al., Science (1992) 256:221-225), thioether forming ligation (Englebretsen, et al., Tet. Letts. (1995) 36(48):8871-8874), hydrazone forming ligation (Gaertner, et al., Bioconj. Chem. (1994) 5(4):333-338), and thiazolidine forming ligation and oxazolidine forming ligation (Zhang, et al., Proc. Natl. Acad. Sci. (1998) 95(16):9184-9189; Tam, et al., WO 95/00846) or by other methods (Yan, L. Z. and Dawson, P. E., “Synthesis of Peptides and Proteins without Cysteine Residues by Native Chemical Ligation Combined with Desulfurization,” J. Am. Chem. Soc. 2001, 123, 526-533; Gieselnan et al., Org. Lett. 2001 3(9):1331-1334; Saxon, E. et al., “Traceless” Staudinger Ligation for the Chemoselective Synthesis of Amide Bonds. Org. Lett. 2000, 2, 2141-2143). Preferred chemical ligation methods employ amide-forming chemical ligation, such as native chemical ligation and extended native chemical ligation.

By “capable of chemical ligation” is intended a moiety that is in a form that can be directly employed in a chemical ligation reaction, or can be converted to a moiety for use in a chemical ligation reaction. In many situations, a moiety capable of chemical ligation will be in a form that must be converted for a ligation reaction to proceed. For instance, when a thioester or selenoester generator of the invention is employed for making a target molecule bearing a n N-terminal amino acid capable of chemical ligation in combination with a C-terminal thioester or selenoester, the N-terminal amino acid is typically protected to avoid intramolecular cyclization or undesired intermolecular condensation with itself. In this way, such a target molecule can be used for a thioester or selenoester-mediated chemical ligation reaction, such as native or extended native chemical ligation, followed by removal of the N-terminal protection for subsequent native or extended native chemical ligation reaction cycles (e.g., sequential native or extended native chemical ligation). In some instances, however, intramolecular cyclization may be desired, which is particularly useful for making cyclic products, such as cyclic peptides. N-terminal amino acids, such as serines, that are capable of being converted to bear an aldehyde moiety by mild oxidation or reductive alkylation is another example, which find particular use in Schiff-base mediated chemical ligation reactions. In other chemical ligation reactions, the N-terminal amino acid can be provided in a ready-to-use chemical ligation form, such as when the N-terminal amino acid bears an azide, halogen, or aminooxy group for other chemical ligation reactions.

Where the N-terminal group comprises an amino acid capable of native or extended native chemical ligation, the amino acid comprises a side chain bearing an atom selected from sulfur and selenium. Examples of amino acids suitable for use in native chemical ligation comprise an alpha-carbon side chain bearing a sulfur or selenium atom, such as cysteine, homocysteine, selenocysteine, homoselenocysteine, and protected forms thereof. Examples of amino acids suitable for use in extended native chemical ligation comprise an alpha-nitrogen side chain bearing a sulfur or selenium atom, which include the alpha-nitrogen substituted 2 or 3 carbon chain alkyl or aryl thiol and selenol auxiliaries, and protected forms thereof as described in Botti et al., WO 02/20557. As can be appreciated, an N-terminal amino acid capable of native or extended native chemical ligation can be protected using a protecting group for the alpha-nitrogen, the side chain sulfur or selenium, or a combination of both, including cyclic protection strategies employing an N-terminal thioproline or extended native chemical ligation alpha-nitrogen substituted auxiliary. The thioester and selenoester generators of the invention preferably employ an amino acid bearing a side chain sulfur or selenium group that is protected.

As described above, the C-terminal group of the thioester and selenoester generators of the invention comprises a thioester and selenoester. This includes any group compatible with the thioester or selenoester group, including, but not limited to, aryl, benzyl, and alkyl groups that may be linear, branched, substituted or unsubstituted, which includes amino acid, peptide and other organic thioester or selenoester moieties. Preferred examples include 3-carboxy-4-nitrophenyl thioesters, benzyl thioesters and selenoesters, mercaptopropionyl thioesters and selenoesters, and mercaptopropionic acid leucine thioesters and selenoesters (See, e.g., Dawson et al., Science (1994) 266:776-779; Canne et al. Tetrahedron Lett. (1995) 36:1217-1220; Kent, et al., WO 96/34878; Kent, et al., WO 98/28434; Ingenito et al., J. Am. Chem. Soc (1999) 121(49):11369-11374; and Hackeng et al., Proc. Natl. Acad. Sci. U.S.A. (1999) 96:10068-10073).

In a preferred embodiment, the C-terminal group comprises a sterically hindered thioester or selenoester having the formula J—CH(R₂)—C(O)—X—R₃, where J comprises a residue of the organic backbone; R₂ comprises any side chain group; X is sulfur or selenium; and R₃ is any thioester or selenoester compatible group; and where one or both of R₂ and R₃ is a group that sterically hinders the thioester or selenoester moiety —C(O)—X—. In a preferred embodiment, one of R₂ and R₃ are selected from a branching group having the formula —C(R₄)(R₅)(R₆), where R₄, R₅, and R₆ each individually are selected from hydrogen and linear, branched, substituted and unsubstituted alkyl, aryl, heteroaryl, and benzyl groups, with the proviso that two or more of R₄, R₅, and R₆ are selected from linear, branched, substituted and unsubstituted alkyl, aryl, heteroaryl, and benzyl groups. The C-terminal group bearing either a sterically hindered or non-hindered thioester or selenoester preferably comprises an amino acid.

By way of example, a preferred thioester and selenoester generator comprising an amino acid synthon having an N-terminal group joined to a C-terminal group through an organic backbone having one or more carbons, comprises the formula:

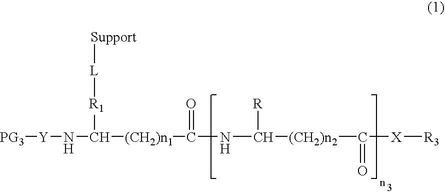

wherein PG₃ is a nucleophile-stable protecting group or is absent; Y is a target molecule of interest that may be present or absent and is lacking reactive functional groups; “Support” is a solid phase, matrix or surface; L is a nucleophile-stable linker; R₁ is a divalent radical lacking reactive functional groups; each R individually is hydrogen or an organic side-chain lacking reactive functional groups; n₁ and n₂ each are from 0 to 2; n₃ is from 0 to 20; X is sulfur or selenium; and R₃ is any group compatible with thioesters or selenoesters.

In compounds of the structure (1), PG₃ is a nucleophile stable protecting group that can be removed under conditions orthogonal to, or the same as the nucleophile stable linker L. Alternatively, PG₃ can be absent. The presence or absence of, and the particular PG₃ employed is chosen based on the N-terminal group of Y. For instance, where the N-terminal group of Y comprises an amino group, such as the alpha amino group of an amino acid, exemplary nucleophile stable amino protecting groups usable for PG₃ include, by way of example, Boc and benzyloxycarbonyl (Cbz) protecting groups, which respectively are removable under mild acidic and mild catalytic hydrogenation conditions. As described above, the N-terminal group may comprise a protected or unprotected amino acid. Where the target molecule of interest is designed as an intermediate for subsequent chemical ligation reactions, a preferred N-terminal amino acid is capable of chemical ligation. Examples of N-terminal amino acids capable of chemical ligation include cysteine residues bearing an N-alpha amino protected with PG₃ or an N-alpha amino protected with PG₃ that is substituted with an auxiliary side chain bearing a thiol or selenol for general or extended native chemical ligation. For N-terminal ligation groups, the thiols, selenols, or other nucleophiles are preferably protected with nucleophile-stable protecting groups such as Acm or benzyl derivatives. Where Y comprises an N-terminal group that is substantially non-reactive, such as a linear, branched, substituted or unsubstituted aliphatic, or other capping group, then PG₃ can be absent, for example, where further elaboration of the support bound target molecule is desired. Alternatively, a reactive group may be present on the N-terminal group, but is generally chosen so as not to react with the C-terminal thioester or selenoester, except where thioester- or selenoester-mediated intramolecular cyclization is desired.

The group Y may comprise any molecule of interest including, for example, an amino acid, peptide, polypeptide, nucleic acid, lipid, carbohydrate, combinations thereof, and the like. Preferred Y groups are peptides.

The linker L may comprise any cleavable group capable of anchoring R₁ to the support material that is stable to nucleophilic conditions. As linker L is stable to nucleophilic conditions, it is cleavable under conditions orthogonal to the conditions for removal of nucleophile-labile protecting groups, such as Fmoc groups.

The use of linker groups in solid phase synthesis is well known, and various linker groups L are usable with the invention. The linker L may be bifunctional, and may serve as a spacer with a cleavable functional group on one end, and a group such as a carboxyl group at the other end that can be activated to allow coupling to a functionalized support material. The linker can be a preformed linker or may be prepared on a support material. Suitable linkers L include, for example, PAL, XAL, PAM, RINK, SCAL, and Sieber-based linker systems as described above. The aforementioned linkers are non-silyl-based linkers or are otherwise lacking a silyl group. Linkers that include a silyl ether group are less preferred, but may be employed in certain embodiments where silyl ether linkages are desired.

Linker L is covalently anchored to a support as described further below. Suitable supports may comprise, for example, matrixes, surfaces, resins or other solid phase or support that is compatible with peptide synthesis or other synthetic schemes associated with the target molecule Y. The support may comprise a functionalized glass, an organic polymer, or other material. Suitable solid supports are described in, for example, “Advanced Chemtech Handbook of Combinatorial & Solid Phase Organic Chemistry,” W. D. Bennet, J. W. Christensen, L. K. Hamaker, M. L. Peterson, M. R. Rhodes, and H. H. Saneii, Eds., Advanced Chemtech, 1998, and elsewhere (See, e.g., G. B. Fields et al., Synthetic Peptides: A User's Guide, 1990, 77-183, G. A. Grant, Ed., W.H. Freeman and Co., New York; NovaBiochem Catalog, 2000; “Synthetic Peptides, A User's Guide,” G. A. Grant, Ed., W.H. Freeman & Company, New York, N.Y., 1992; “Principles of Peptide Synthesis, 2nd ed.,” M. Bodanszky, Ed., Springer-Verlag, 1993; “The Practice of Peptide Synthesis, 2nd ed.,” M. Bodanszky and A. Bodanszky, Eds., Springer-Verlag, 1994; “Fmoc Solid Phase Peptide Synthesis, A Practical Approach,” W. C. Chan and P. D. White, Eds., Oxford Press, 2000).

The group R₁ may comprise any organic divalent radical that is lacking a reactive functional group. Thus, an organic divalent radical that is lacking a reactive functional group refers to divalent radicals in which such reactive functional groups are entirely absent, as well as divalent radicals that contain protected functional groups that would otherwise be reactive but for the presence of the protecting group(s). Where R₁ includes a divalent radical containing one or more protected functional groups, the protecting group can be removable under conditions orthogonal to other protecting groups that may be present on the organic backbone, and/or PG₃. In a preferred embodiment, an R₁ protecting group is removable under the same or similar conditions that result in cleavage of the linker L. For example, in most instances, R₁ will have a functional group that is protected by its covalent attachment to linker L, where linker L provides the appropriate protection, and where cleavage of linker L results in simultaneous release from the support and deprotection of R₁.

In a preferred embodiment, the R₁ group comprises a side chain of an amino acid selected from aspartic acid, glutamic acid, glutamine, lysine, serine, threonine, arginine, cysteine, histidine, tryptophan, tyrosine, and asparagine. Thus, the group R₁, in many embodiments, will comprise a radical based on an amino acid side chain or derivative thereof that has a functionality capable of covalently binding to the linker L. Thus, for example, the group R₁ may comprise the radical

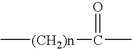

wherein n is 1 (corresponding to aspartic acid and asparagine) or n is 2 (corresponding to glutamic acid and glutamine),

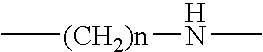

wherein n is 4 (lysine),

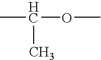

(threonine),

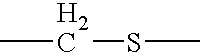

(cysteine),

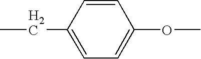

(tyrosine),

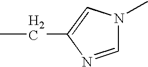

(proline),

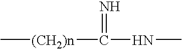

wherein n is 3 (arginine),

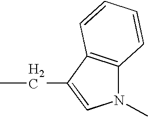

(tyrosine), or other radical associated with an amino acid side chain. The above examples represent some of the side chain functionalities associated with common, naturally occurring amino acids, and are only exemplary. Numerous other divalent radicals suitable for R₁, including side chains of less common amino acids and synthetic or modified amino acids, will suggest themselves to those skilled in the art and may also be used.

The term “organic group” and “organic radical” as used herein means a hydrocarbon group that is classified as an aliphatic group, cyclic group, aromatic group, functionalized derivatives thereof, and/or various combination thereof. The term “aliphatic group” means a saturated or unsaturated linear or branched hydrocarbon group and encompasses alkyl, alkenyl, and alkynyl groups, for example. The term “alkyl group” means a saturated linear or branched hydrocarbon group including, for example, methyl, ethyl, isopropyl, t-butyl, heptyl, dodecyl, octadecyl, amyl, 2-ethylhexyl, and the like. The term “alkenyl group” means an unsaturated, linear or branched hydrocarbon group with one or more carbon-carbon double bonds, such as a vinyl group. The term “alkynyl group” means an unsaturated, linear or branched hydrocarbon group with one or more carbon-carbon triple bonds. The term “cyclic group” means a closed ring hydrocarbon group that is classified as an alicyclic group, aromatic group, or heterocyclic group. The term “alicyclic group” means a cyclic hydrocarbon group having properties resembling those of aliphatic groups. The term “aromatic group” or “aryl group” means a mono- or polycyclic aromatic hydrocarbon group. The term “heterocyclic group” means a closed ring hydrocarbon in which one or more of the atoms in the ring is an element other than carbon (e.g., nitrogen, oxygen, sulfur, etc.). The organic groups may be functionalized or otherwise comprise additional functionalities associated with the organic group, such as carboxyl, amino, hydroxyl, and the like, which may be protected or unprotected. For example, the phrase “alkyl group” is intended to include not only pure open chain saturated hydrocarbon alkyl substituents, such as methyl, ethyl, propyl, t-butyl, and the like, but also alkyl substituents bearing further substituents known in the art, such as hydroxy, alkoxy, alkylsulfonyl, halogen atoms, cyano, nitro, amino, carboxyl, etc. Thus, “alkyl group” includes ether groups, haloalkyls, nitroalkyls, carboxyalkyls, hydroxyalkyls, sulfoalkyls, etc.

The group R may comprise hydrogen or any organic side-chain lacking reactive functional groups. In this regard, R may comprise an amino acid side chain, with the amino acid glycine corresponding to the case where R comprises hydrogen. Where R is a side chain associated with an amino acid that has a reactive functionality on the side chain such as glutamic acid, a suitable protecting group or groups may be used so that R is lacking a reactive functional group as described above for R₁. Alternatively, R may bear a functional group that is otherwise substantially unreactive under the conditions employed in a given synthesis step of interest. Such substantially unreactive functional groups can include primary or secondary alcohols, or aminooxy or ketone moieties, for example, when cycles of activation, acylation, and deprotection procedures are employed in peptide synthesis. It will be appreciated that protecting groups for R, as well as each R can vary independently with each component bearing such R group in the organic backbone.

The group R₃ may comprise any group that is compatible with a thioester or selenoester. Exemplary R₃ groups comprise, for example, alkyl, aryl, and benzyl groups, including phenyl, t-butyl, and ethyl carboxy alkylate groups. Such R₃ groups may also comprise amino acids and peptides, and other organics. Various activated thioesters and selenoesters are known, and suitable divalent radicals associated with such thioester and selenoesters are employable, and may be used with the invention.

Compounds of the structure (1) represent a variety of intermediates usable for thioester and selenoester generation. As described above, where Y is absent and where n₃ is zero, the structure (1) corresponds to a single amino acid bound to linker L via side chain radical R₁. Where n, is zero, the amino acid is an alpha-amino acid, and where n₁ is 1 or 2, the amino acid correspondingly comprises a β-amino acid or a γ-amino acid. Where n₃ is 1, 2, 3, or higher and Y is absent, the compound (1) corresponds respectively to a dipeptide, tripeptide, and tetrapeptide or higher peptide, which may comprise alpha, beta, and gamma amino acids respectively where n₂ is 0, 1, or 2. In a preferred embodiment, n₃ is from 0 to 15, with 0 to 10, 0 to 5, 0 to 3, 0 to 2, and 0 to 1 being the most preferred in this order. Where Y is present, the compound (1) may comprise a longer peptide, a peptide-polymer conjugate, or other peptide or polypeptide compound as described above.

In another preferred embodiment, and by way of example, a preferred sterically hindered thioester and selenoester generator comprising an amino acid synthon having an N-terminal group joined to a C-terminal group through an organic backbone having one or more carbons, comprises the formula:

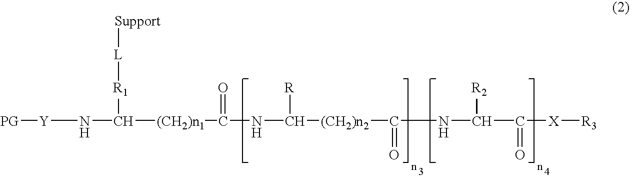

where PG is a protecting group that may be present or absent; Y is a target molecule of interest that may be present or absent and is lacking functional reactive groups; L is a nucleophile-stable linker; Support is a solid phase, matrix or surface; R₁ is a divalent radical lacking reactive functional groups; each R and R₂ individually is any side chain group and may be the same or different and are lacking functional reactive groups; n₁ and n₂ each individually is 0, 1, or 2; n₃ is 0 to 20; n₄ is 0 or 1; X is sulfur or selenium; and R₃ is any thioester or selenoester compatible group; and wherein one or more of R₂ and R₃ is a group that sterically hinders the thioester or selenoester moiety —C(O)—X—.

In the compounds of the structure (2), the Y, L, R₁, and R groups are the same as described above for compounds of the structure (1). In the structure (2), protecting group PG may be any protecting group, including nucleophile-stable and nucleophile-labile protecting groups, and may be present or absent. In structure (2), an additional C-terminal alpha amino acid may optionally be present with a group R₂, which may comprise hydrogen or any organic side chain group. As with structure (1), n₃ in structure (2) preferably is from 0 to 20, 0 to 15, with 0 to 10, 0 to 5, 0 to 3, 0 to 2, and 0 to 1 being the most preferred in this order, i.e., 0 to 1 being most preferred. In the compounds of structure (2), at least one of the groups R₂ and R₃ is a group that sterically hinders the —C(O)—X—moiety. The terms “sterically hindering” and “sterically hindered” as used herein refers to a group or groups that prevent or help prevent hydrolysis or self-induced aminolysis associated with the —C(O)—X—moiety. The sterically hindering group R₂ and/or R₃ additionally aids in preventing racemization of the carbon bound to the R₂ group where n₄ is 1. Where n₂ and n₄ are 0 and n₃ is greater than 0, the sterically hindering R₃ group prevents racemization associated with the carbon bound to the R group and, where n₁, n₂, n₃, and n₄ each are 0, the sterically hindering R₃ group prevents racemization associated with the carbon bound to the R₁ group joined to the linker L.

Sterically hindering groups usable for R₂ and/or R₃ include, by way of example, branched alkane, cycloalkane, alkyl-substituted aryl, and heteroaryl groups, and combinations thereof. Such sterically hindering groups may comprise the formula —C(R₄)(R₅)(R₆), or as alternatively presented:

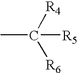

where R₄ comprises hydrogen, a linear, branched, cyclic substituted or unsubstituted alkyl, aryl, heteroaryl, or benzyl group, and R₅ and R₆ each individually comprise a linear, branched, cyclic substituted, or unsubstituted alkyl, aryl, heteroaryl, or benzyl group. Other groups providing steric hindrance for the thioester or selenoester moiety may also be used for group R₂ and/or R₃.

The use of the aforementioned protecting groups, linkers, and solid phase supports, as well as specific protection and deprotection reaction conditions, linker cleavage conditions, use of scavengers, and other aspects of solid phase peptide synthesis are well known and are also described in “Protecting Groups in Organic Synthesis,” 3rd Edition, T. W. Greene and P. G. M. Wuts, Eds., John Wiley & Sons, Inc., 1999; NovaBiochem Catalog, 2000; “Synthetic Peptides, A User's Guide,” G. A. Grant, Ed., W.H. Freeman & Company, New York, N.Y., 1992; “Advanced Chemtech Handbook of Combinatorial & Solid Phase Organic Chemistry,” W. D. Bennet, J. W. Christensen, L. K. Hamaker, M. L. Peterson, M. R. Rhodes, and H. H. Saneii, Eds., Advanced Chemtech, 1998; “Principles of Peptide Synthesis, 2nd ed.,” M. Bodanszky, Ed., Springer-Verlag, 1993; “The Practice of Peptide Synthesis, 2nd ed.,” M. Bodanszky and A. Bodanszky, Eds., Springer-Verlag, 1994; “Protecting Groups,” P. J. Kocienski, Ed., Georg Thieme Verlag, Stuttgart, Germany, 1994; “Fmoc Solid Phase Peptide Synthesis, A Practical Approach,” W. C. Chan and P. D. White, Eds., Oxford Press, 2000, G. B. Fields et al., Synthetic Peptides: A User's Guide, 1990, 77-183, and elsewhere.

### Methodology for Synthesis of Thioester and Selenoester Generators

The thioester and selenoester generators of the invention can be prepared by providing a precursor composition having a free C-terminal carboxyl, followed by conversion of the free carboxyl to a thioester or selenoester to form the desired thioester or selenoester generator. In particular, the precursor composition includes an amino acid synthon having an N-terminal group joined to a C-terminal free carboxyl through an organic backbone that comprises a carbon having a side chain anchored to a support through a nucleophile-stable linker. The organic backbone lacks reactive functional groups and the N-terminal group can be unprotected or protected, depending on the intended end use.

When a non-sterically hindered thioester or selenoester is desired, the N-terminal group is unprotected, or protected with a nucleophile-stable protecting group. Presence of a nucleophile-stable protecting group permits removal of the protecting group under non-nucleophilic conditions (i.e., in the presence of the formed thioester or selenoester), without destroying the thioester or selenoester moiety. When the N-terminal group is unprotected, it is preferred to be group that is substantially non-reactive under conditions for carboxyl activation and coupling of a thioester or selenoester component. When-a sterically hindered thioester or selenoester is desired, the N-terminal group may be protected or unprotected. In this situation, the protecting group can be nucleophile-stable or -labile. For an unprotected N-terminus, here again it is preferred that the N-terminus bears a group that is substantially non-reactive under conditions for carboxyl activation and coupling of a thioester or selenoester component.

Conversion of the free carboxylate of the precursor composition to the thioester or selenoester involves contacting an activated form of the free carboxyl with a compound selected from a thiol moiety, a selenol moiety, a preformed thioester, and a preformed selenoester. Activation of the free carboxyl can be carried out by any number of activating agents that are capable of forming a carboxyester. Preferred carboxyl activation techniques include in situ activation and/or the use of preformed activated amino acid derivatives such as commercially available pentafluorophenyl (OPfp) activated esters. Activating reagents capable of providing in situ generation of activated carboxyesters (OAct) include, by way of example, Obt (benzotriazoly carboxy ester) and OAt (azabenzotriazoly carboxy ester) activation reagents such as DIC/HOBt, HATU, PyBOP, PyAOP, TBTU, HBTU, and like activation systems. Other activation reagents, such as TFFH (acid fluoride activation), may also be used. Activation can be carried out in the presence of thiol moiety, a selenol moiety, a preformed thioester, and a preformed selenoester, or can be provided in a pre-activated form followed by the addition of the thiol moiety, a selenol moiety, a preformed thioester, and a preformed selenoester. An advantage of the former approach is a reduction in overall reaction time, which reduces potential for racemization or other unwanted side-reactions.

In a preferred embodiment, the compound bearing the thiol or selenol moiety used in the thioester or selenoester conversion process comprises the formula HS—R₃ or HSe—R₃. The R₃ group is as defined above, and may be any group compatible with thioesters or selenoesters. This includes linear, branched, substituted and unsubstituted alkyl, aryl, heteroaryl, and benzyl groups. For example, mercaptans and senenols, such as mercaptoproprionic acid, mercaptoproprionyl, thiophenol, selenophenol, selenolproprionic acid, and selenolproprionyl compounds can be used for this purpose.

The preformed thioester or selenoester compounds employed for conversion and formation of the thioester and selenoester generators preferably comprise an amino acid or peptide. This includes preformed thioester or selenoester compounds of the formula H[NH—C(R₂)—C(O)]ₙ₅—S—R₃; and H[NH—C(R₂)—C(O)]ₙ₅—Se—R₃; where R₂ and R₃ are as defined above, and each individually are the same or different and are lacking reactive functional groups; where n₅ is from 1 to 5, with n₅ preferably being from 1 to 4, with 1 to 3, 1 to 2, and 1 being the most preferred in this order. For example, chemically synthesized thioester and selenoester amino acids and peptides can be made from the corresponding α-thioacids or α-selenoacids, which in turn, can be synthesized on a thioester- or selenoester resin or in solution, although the resin approach is preferred. The α-thioacids or selenoacids can be converted to the corresponding 3-carboxy-4-nitrophenyl thioesters or selenoesters, to the corresponding benzyl ester, or to any of a variety of alkyl thioesters or selenoesters. As another example, a trityl-associated mercaptoproprionic acid leucine thioester- or selenoester generating resin can be utilized (Hackeng et al., supra). Thioester and selenoester synthesis also can be accomplished using a 3-carboxypropanesulfonamide safety-catch linker by activation with diazomethane or iodoacetonitrile followed by displacement with a suitable thiol or selenol (Ingenito et al., supra; Shin et al., J. Am. Chem. Soc. (1999) 121:11684-11689). Various other synthetic approaches for making preformed thio- or selenoesters may be employed as well (e.g., Beletskaya et al., Mendeleev Commun. (2000) 10(4):127-128; Kim et al., J. Chem. Soc., Chem. Commun. (1996) 1335; Dowd et al., J. Am. Chem. Soc. (1992) 114:7949; Wang et al., Synthetic Comm. (1999) 29(18):3107-3115; Lu et al., Synthetic Comm. (1999) 29(2):219-225; and Kozikowski et al., Tetrahedron (Symposium Series) (1985) 41:4821-4834).

The sterically hindered thioester and selenoester generators of the invention may be prepared by converting the free carboxyl of the precursor composition to a sterically hindered thioester or selenoester. This can be accomplished by coupling a compound comprising a sterically hindered thiol or selenol moiety to an activated form of the free carboxyl. In a preferred embodiment, the sterically hindered thiol or selenol moiety comprises the formula X—C(R₄)(R₅)(R₆), or as alternatively presented:

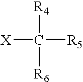

where X is thiol or selenol; and R₄, R₅, and R₆ each individually are selected from the group consisting of hydrogen and linear, branched, substituted and unsubstituted alkyl, aryl, heteroaryl, and benzyl groups, with the proviso that two or more of R₄, R₅, and R₆ are selected from the group consisting of linear, branched, substituted and unsubstituted alkyl, aryl, heteroaryl, and benzyl groups.

The sterically hindered thioester and selenoester generators also may be prepared using preformed sterically hindered thioester or selenoesters. This process involves converting the free carboxyl group to a sterically hindered thioester or selenoester by coupling a preformed amino acid or peptide having a sterically hindered thioester or selenoester to form an amide bond therein between. In this instance, the amino acid or peptide thioester or selenoester comprises an unprotected N-terminal amine and a sterically hindered C-terminal thioester or sterically hindered selenoester.

Sterically hindered thioester and selenoester generators also may be prepared by converting a sterically hindered C-terminal carboxyl group to a thioester or selenoester. A sterically hindered C-terminal carboxyl group for this purpose comprises the formula:

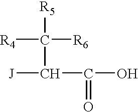

where J comprises a residue of the organic backbone; R₄, R₅, and R₆ each individually are any side chain lacking a reactive functional group and are selected from the group consisting of hydrogen and linear, branched, substituted and unsubstituted alkyl, aryl, heteroaryl, and benzyl groups, with the proviso that two or more of R₄, R₅, and R₆are selected from the group consisting of linear, branched, substituted and unsubstituted alkyl, aryl, heteroaryl, and benzyl groups. Conversion of the sterically hindered C-terminal carboxylate to a sterically hindered thioester or selenoester may be carried out in combination with non-hindered thiols, selenols, preformed thioesters, and preformed selenoesters, as well as sterically hindered versions thereof.

In a preferred embodiment, and by way of example, a preferred method for producing a thioester and selenoester generator comprising an amino acid synthon having an N-terminal group joined to a C-terminal group through an organic backbone having one or more carbons is carried out as follows. First, a precursor composition is provided having the formula:

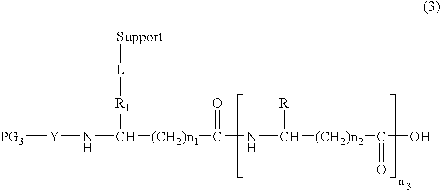

where PG₃, Y, R₁, L, Support, R, n₁, n₂, and n₃ are as defined above for structure (1). The free carboxyl of structure (3) is then converted to a thioester or selenoester to form a thioester or selenoester generator having the formula:

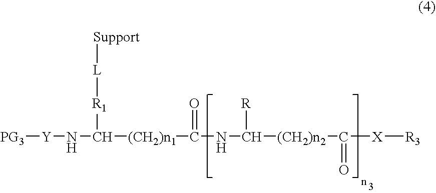

where X is sulfur or selenium; and R₃ is as defined above for structure (1).

In another preferred embodiment, and by way of example, a preferred method for producing a sterically hindered thioester and selenoester generator comprising an amino acid synthon having an N-terminal group joined to a C-terminal group through an organic backbone having one or more carbons is carried out as follows. First, a precursor composition is provided having the formula:

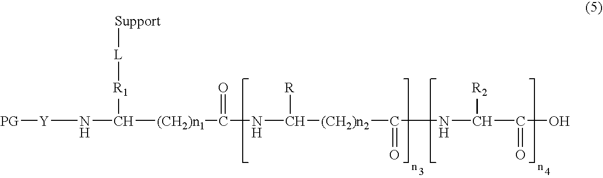

where PG, Y, R₁, L, Support, R, R₂, n₁, n₂, n₃ and n₄ are as defined above for structure (2). The free carboxyl of structure (5) is then converted to a sterically hindered thioester or selenoester to form a sterically hindered thioester or selenoester generator having the formula:

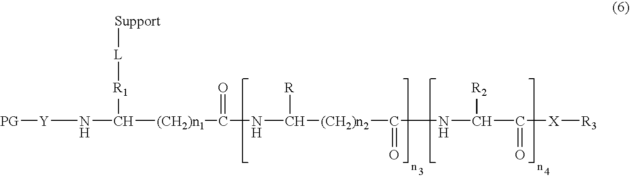

where X is sulfur or selenium; and R₂ and R₃ is as defined above for structure (2).

The activation of carboxyl groups as described above, as well protection and deprotection and linker cleavage protocols, and solid-phase peptide synthesis generally are also described in “Protecting Groups in Organic Synthesis,” 3rd Edition, T. W. Greene and P. G. M. Wuts, Eds., John Wiley & Sons, Inc., 1999; NovaBiochem Catalog, 2000; “Synthetic Peptides, A User's Guide,” G. A. Grant, Ed., W.H. Freeman & Company, New York, N.Y., 1992; “Advanced Chemtech Handbook of Combinatorial & Solid Phase Organic Chemistry,” W. D. Bennet, J. W. Christensen, L. K. Hamaker, M. L. Peterson, M. R. Rhodes, and H. H. Saneii, Eds., Advanced Chemtech, 1998; “Principles of Peptide Synthesis, 2nd ed.,” M. Bodanszky, Ed., Springer-Verlag, 1993; “The Practice of Peptide Synthesis, 2nd ed.,” M. Bodanszky and A. Bodanszky, Eds., Springer-Verlag, 1994; “Protecting Groups,” P. J. Kocienski, Ed., Georg Thieme Verlag, Stuttgart, Germany, 1994; “Fmoc Solid Phase Peptide Synthesis, A Practical Approach”, W. C. Chan and P. D. White, Eds., Oxford Press, 2000, G. B. Fields et al., Synthetic Peptides: A User's Guide, 1990, 77-183, and elsewhere, as noted above.

The thioester and selenoester generators of the invention also can be prepared by a nucleophile-based synthesis scheme. This is particularly useful where nucleophiles are employed in the synthesis of a target molecule of interest, such as a peptide or polypeptide prepared by Fmoc- or Nsc-SPPS. The method may be employed to make sterically hindered and non-hindered thioesters and selenoesters. The method involves, in one embodiment, the following steps (a) through (e).

Step (a) First, a composition is provided that comprises an amino acid synthon having an N-terminal group joined to a C-terminal group through an organic backbone comprising one or more carbons. The N-terminal group of the composition comprises a reactive functional group protected with a nucleophile-labile protecting group, and the C-terminal group comprises a carboxyl protected with a carboxyl protecting group removable under conditions orthogonal to the nucleophile-labile protecting group. The organic backbone is lacking reactive functional groups and comprises a carbon having a side chain anchored to a support through a nucleophile-stable linker cleavable under conditions orthogonal to the carboxyl protecting group. Thus, the linker and the nucleophile-labile and carboxyl protecting group pairing employed in Step (a) are removable under orthogonal conditions, and the carboxyl protecting group is stable to the conditions employed for removal of the N-terminal nucleophile-labile protecting group. The organic backbone may also comprise a target molecule of interest, or portion thereof.

As described above, the preferred support is compatible with solid-phase organic synthesis (SPOS) or solid-phase peptide synthesis (SPPS). The preferred nucleophile-stable linkers are removable under acidic conditions as provided by trifluoracetic acid (TFA) or hydrogen fluoride (HF), under catalytic conditions in the presence of H₂, or by other mechanism such as light (e.g., UV photolysis). The amino acid synthon will preferably be composed of an amino acid having a side chain anchored to the support through the linker, and may be provided in the initial composition as a single amino acid residue, peptide, or an organic composition containing an amino acid component, peptide or residue thereof. As also noted above, the organic backbone is lacking reactive functional groups. In most instances, protecting groups, if present on the organic backbone, are preferably selected so as to be removable under the same conditions as the linker. However, protecting groups can be selected that provide an additional level of orthogonality when site-specific modifications to the organic backbone are desired during or after synthesis.

For the C-terminal group, exemplary carboxyl protecting groups removable under conditions orthogonal to the nucleophile-labile protecting group are Allyl and ODmab. Allyl groups are stable to nucleophiles, yet are removable by palladium-catalyzed hydrogenation. ODmab groups can be removed with hydrazine, which is a very strong nucleophile, but are stable to typical conditions employed for removal of most other nucleophile-labile protecting groups, such as N-terminal amino protecting groups Fmoc and 2-(4-nitrophenylsulfonyl)ethoxycarbonyl (Nsc Bzi). For instance, Fmoc and Nsc groups are readily removed by piperidine, which is a much weaker nucleophile compared to hydrazine. This difference in stability provides the appropriate level of orthogonality.

Depending of the N-terminal functional group, various nucleophile-labile protecting groups may be employed, such as nucleophile-labile amino protecting groups where the N-terminal functional group is an amine, e.g., Fmoc and Nsc. As can be appreciated, other nucleophile-labile and carboxyl protecting groups having compatible orthogonality as described may also be employed in Step(a).

By way of example, preferred compositions employable in Step (a) comprise the formula:

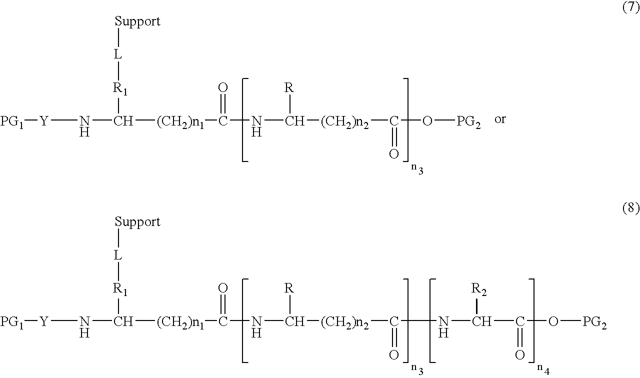

Referring to structures (7) and (8), PG₁ is a nucleophile-labile protecting group; Y is a target molecule of interest that may be present or absent; L is a nucleophile-stable linker; R₁ is a divalent radical lacking reactive functional groups; each R and R₂ individually is hydrogen or any organic side-chain lacking reactive functional groups; n₁ and n₂ each are from 0 to 2; n₃ is from 0 to 20; n₄ is 0 to 1; and PG₂ is any protecting group that is removable under conditions orthogonal to removal of PG₁ and cleavage of L. Y, R₁, L, Support, R, R₂, and n₁, n₂, n₃, and n₄ are as described above for the structure (2), with the proviso that Y, R₁, L, Support, R, R₂, are compatible with nucleophile-based SPOS and/or SPPS.

The protecting group PG₁ may comprise any of a variety of nucleophile-labile protecting groups. As noted above, the particular protecting group PG₁ may be selected based on the particular molecule of interest or target molecule, compatibility with other protecting groups or functionalities that will be present during synthesis, or other considerations. The protecting group PG₂ may comprise any group capable of protecting a carboxyl group and is orthogonal to the nucleophile-labile protecting group PG₁ and the nucleophile-stable linker L, as discussed above. Exemplary protecting groups PG₂ and PG₁ fitting these criteria include allyl and ODmab groups for the C-terminal carboxyl protection, Fmoc and Nsc when the N -terminal group is an amine, and where a suitable linker would be one cleavable under acidic conditions.

Compositions of the structures (7) and (8) are easily extensible using conventional Fmoc-based or Nsc-based solid-phase organic or peptide synthesis (i.e., SPOS or SPPS) techniques, and provide for a “side chain”-based anchoring during synthesis for elaborating a target molecule of interest Y. For instance, structures (7) and (8) can be employed in a variety of nucleophile-based chain elongation synthesis schemes involving repeated cycles of nucleophilic deprotection and coupling with incoming compounds bearing a reactive moiety and PG₁, as illustrated below for structure (7).

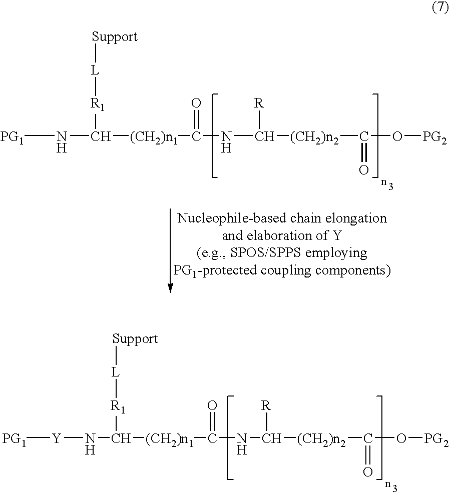

Step (b) From a composition provided in Step (a), the nucleophile-labile protecting group is then selectively removed under nucleophilic conditions to form an N-terminal group comprising a first reactive functional group. For instance, where PG₁ is a nucleophile-labile amino protecting group, and the pendant N-terminal group of Y is an amine, PG₁ can be Fmoc or Nsc, and removal thereof can be carried out under basic conditions that do not remove PG₂.

By way of example, preferred compositions generated in Step (b) comprise the formula:

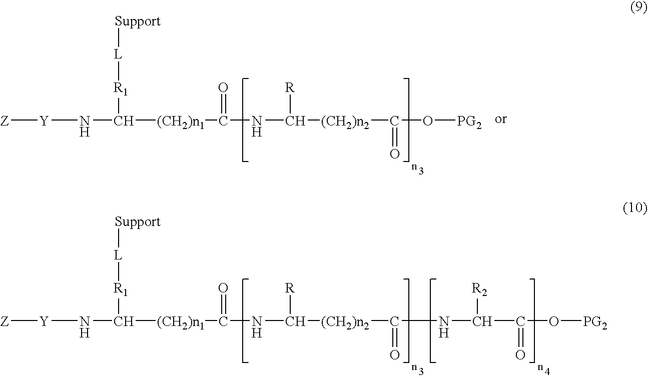

where Y, R₁, L, Support, R, R₂, n₁, n₂, n₃, and n₄ are as defined above for structure (2), with the proviso that Y, R₁, L, Support, R, R₂ are compatible with nucleophile-based SPOS and/or SPPS; and Z comprises a reactive functional group of interest.

Step (c) Following removal of the nucleophile-labile protecting group in Step (b), the deprotected N-terminal reactive functional group of the product of Step (b) is coupled to a compound of interest. The compound of interest bears a single reactive moiety capable of forming a covalent bond with the N-terminal reactive functional group. Various compounds can be employed in this step, depending on the intended end use, to generate an elongated product having the compound of interest on the N-terminal group.

In one embodiment of Step(c) hereinafter referred to as Step (c-i), an unprotected compound may be used for the coupling in Step(c). As such, the unprotected compounds will bear a single reactive moiety capable of forming a covalent bond with the N-terminal reactive functional group. Preferred unprotected compounds for Step (c-i) are those that are substantially unreactive in the presence of carboxyl activation agents and thiols or selenols, i.e., conditions employed for nucleophile-based synthesis of the thioester or selenoester. Examples of suitable unprotected compounds for Step (c-i) include mono-functionalized compounds that are missing other functional reactive groups, or have additional functional groups that are substantially unreactive under conditions employed for nucleophile-based synthesis of the thioester or selenoester, such as mono-functionalized amino acids, peptides, and other organics in which all but the single reactive moiety are capped, monofunctionalized conjugates, dyes, fluorescent labels or tracers, radioactive elements, metal chelators, and the like, as well as mono-functionalized alkyls, aryls, benzyls, polymers, and the like. Unprotected compounds for Step (c-i) having additional functional groups that are substantially unreactive under conditions employed for nucleophile-based synthesis of the thioester or selenoester, include, for example, alcohols and ketones. Unprotected compounds for Step (c-i) may also include bi-functional moieties (e.g., diacids and diamines), or moieties that generate a new reactive functional group following coupling (e.g., amino and acid anhydrides). For the bifunctional moieties, the newly generated functionally group will typically require capping or protection prior to subsequent thioesterification or selenoesterfication.

In another embodiment of Step (c), hereinafter referred to as Step (c-ii), an amino-protected compound (c-ii) can be used for the coupling. In this situation, the amino-protected compound of Step (c-ii) comprises a single reactive moiety capable of forming a covalent bond with the N-terminal reactive functional group, and bears an amino group that is protected with a nucleophile-stable amino protecting group removable under conditions orthogonal to removal of the carboxyl protecting group. Thus, such amine-protected compounds of Step (c-ii) lack reactive functional groups other than a single reactive moiety that forms the covalent bond with the N-terminal reactive functional group of the product of Step (b). Examples of suitable amino-protected compounds of Step (c-ii) include amino acids, peptides, and other organics possessing an amino functionality. Monoamines, diamines, or higher amines are other examples.

In yet another embodiment of Step (c), hereinafter referred to as Step (c-iii), the coupling in may be carried out with a protected compound having a single reactive moiety that forms a covalent bond with the N-terminal reactive functional group of the product of Step (b), and one or more additional reactive functional groups protected with a protecting group that is removable under conditions orthogonal to removal of the carboxyl protecting group. Protected compounds for Step (c-iii) are particularly useful for forming sterically hindered thioesters or selenoesters. Preferred examples of protected compounds for Step (c-iii) include amino acids and peptides, and other organic compounds having more than one reactive functional group, and include the amine-protected compounds for Step (c-iii).

By way of example, preferred compositions generated in Step (c) comprise the formula:

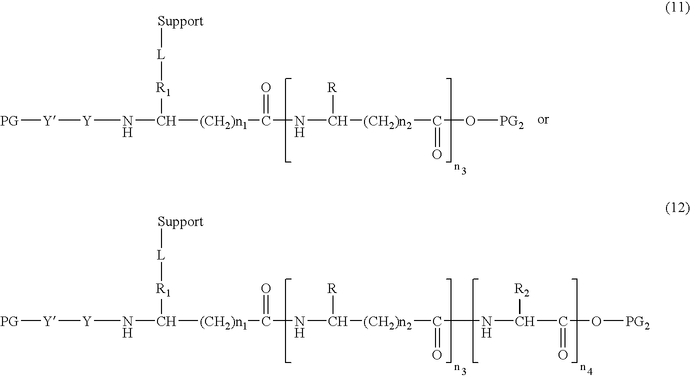

where Y; R₁, L, Support, R, R₂, n₁, n₂, n₃, and n₄ are as defined above for structure (2), with the proviso that Y; R₁, L, Support, R, R₂ are compatible with nucleophile-based SPOS and/or SPPS. Referring to structure (11), Y′ is a compound of interest lacking reactive functional groups; and PG may be present or absent, with the proviso that when present, PG is a nucleophile-stable amino protecting group removable under conditions orthogonal to PG₂ and Y′ bears an N-terminal amino group that is protected by PG. Referring to structure (12), Y′ is a compound of interest lacking reactive functional groups; and PG may be present or absent, with the proviso that PG is removable under conditions orthogonal to PG₂.

Step (d) Following the coupling of a compound of interest to the deprotected N-terminal reactive functional group in Step (c), the C-terminal carboxyl protecting group of that product is selectively removed to generate a free carboxyl group. Conditions for removing the carboxyl protecting group are chosen based on the protecting group employed. For instance, where an ally group is employed, palladium-catalyzed hydrogenation can be used, or where an ODmab group is employed, the appropriate hydrazine cocktail can be used.

By way of example, preferred compositions generated in Step (d) comprise the formula:

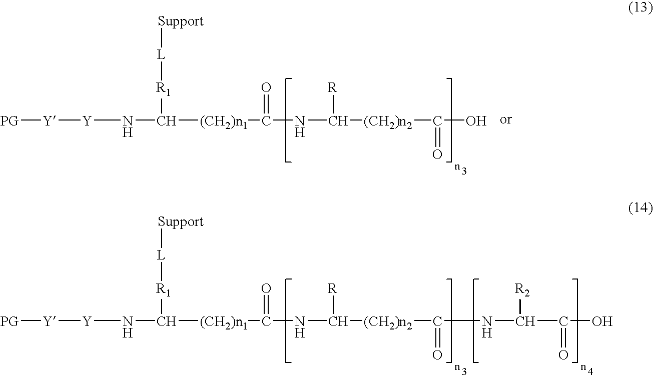

where PG, Y; R₁, L, Support, R, R₂, n₁, n₂, n₃, and n₄ are as defined above for structure (2) and Y′ is as defined in structure (11).

Step (e) Following the selective removal of the C-terminal carboxyl protecting group, and generation of a free carboxyl group in Step (d), the free carboxyl group of the product of Step (d) is converted to a thioester or selenoester. The type of thioester or selenoester formed can vary depending on the compound of interest employed in the coupling step, and thus the compound present on the N-terminus of the product generated in Step (d).

In particular, it is preferable to covert the free carboxyl of the product of Step (d) to a sterically hindered thioester or selenoester when the product of Step (d) bears a protected compound from Step (c-iii) on its N-terminus, i.e., a protected compound having one or more reactive functional groups protected with a protecting group removable under conditions orthogonal to the carboxyl protecting group employed in Steps (a)-(e), regardless of the type of protecting group(s) present on the protected compound of interest. For instance, an exemplary protected compound from Step (c-iii) is any amino acid protected with any number of different protecting groups, including amino protecting groups removable under nucleophilic conditions, such as Fmoc or Nsc. In this situation, a sterically hindered thioester or selenoester moiety can provide some protection against nucleophilic cleavage if one desires to remove the nucleophile-labile protecting group in the presence of the thioester or selenoester, particularly where non-nucleophilic bases are employed. In most cases, however, a protected compound coupled in Step (c) will bear a nucleophile-stable protecting group.

Conversion of the free carboxyl group of a product of Step (d) that is formed with an unprotected compound of Step (c-i) or amine-protected compound of Step (c-iii) may be carried out to generate either sterically hindered or non-hindered thioesters or selenoesters.

As described above, a preformed thioester or selenoester, or compounds bearing a thiol or selenol moiety, may be coupled to an activated form of the free carboxyl of the product of Step (d) to convert, and thus generate the desired thioester or selenoester.

By way of example, preferred compositions generated in Step (e) comprise the formula:

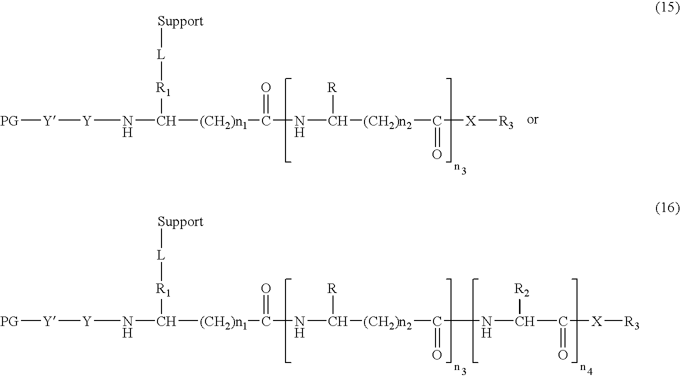

Referring to structure (15), Y, R₁, L, Support, R, n₁, n₂, and n₃ are as defined above for structure (1); Y′ is as defined in structure (11), PG may be present or absent and comprises a nucleophile-stable protecting group; and X is sulfur or selenium; and R and R₃ are as defined above for structure (2). Referring to structure (16), Y, R₁, L, Support, R, n₁, n₂, and n₃ are as defined above for structure (2); Y′ is as defined for structure (12), PG may be present or absent; X is sulfur or selenium; and R₂ and R₃ are as defined above for structure (2).

At this stage, the support-bound thioester or selenoester product can be further elaborated or modified, for example, by on-support modifications to the organic backbone/target molecule of interest. In most instances any additional elongation or modifications are preferably those that do not damage the thioester or selenoester moieties. For example, where the N-terminal group bears a protecting group removable under non-nucleophilic conditions, it is possible to carry out one or more additional cycles of SPOS or SPPS using a non-nucleophilic synthesis scheme, e.g., Boc-SPPS. Coupling of additional reactive groups, which are generally unstable to nucleophilic cycles of chain elongation, carboxyl activation, thioester, or selenoester formation, can be performed at this stage. This is particularly useful when one desires to modify the N-terminus with a functional group such as an aldehylde, acid, conjugate group, or other group or structure. As another example, side chains of the organic backbone/target molecule of interest that were chosen to be orthogonal to reagents and conditions employed in Steps (a)-(e), and are removable under conditions orthogonal to the linker, can be removed and those side chains modified. It also may desirable to generate cyclic forms of the product of Step (e) while still bound to the support. This may be accomplished where the pendant N-terminal group bears, for example, a functional group reactive with thioesters or selenoester that is protected with a protecting group removable under conditions orthogonal to the linker and compatible with thioesters or selenoesters (e.g., an Acm-protected N-terminal Cysteine). Thus, once the N-terminal protecting group is removed, the support-bound material can form a cyclic product.

The organics, equipment, supports, amino acids, diversity components, linkers, and protecting groups finding use in the above nucleophile-based method can be obtained from a variety of commercial sources, prepared de novo, or a combination thereof. Moreover, the reagents and other materials employed for the method, as well as alternative components will be apparent to one of ordinary skill in the art (See, e.g., “Protecting Groups in Organic Synthesis,” 3rd Edition, T. W. Greene and P. G. M. Wuts, Eds., John Wiley & Sons Inc., 1999; NovaBiochem Catalog, 2000; “Synthetic Peptides, A Users Guide,” G. A. Grant, Ed., W.H. Freeman & Company, New York, N.Y., 1992; “Advanced Chemtech Handbook of Combinatorial & Solid Phase Organic Chemistry,” W. D. Bennet, J. W. Christensen, L. K. Hamaker, M. L. Peterson, M. R. Rhodes, and H. H. Saneii, Eds., Advanced Chemtech, 1998; “Principles of Peptide Synthesis, 2nd ed.,” M. Bodanszky, Ed., Springer-Verlag, 1993; “The Practice of Peptide Synthesis, 2nd ed.,” M. Bodanszky and A. Bodanszky, Eds., Springer-Verlag, 1994; “Protecting Groups,” P. J. Kocienski, Ed., Georg Thieme Verlag, Stuttgart, Germany, 1994, “Fmoc Solid Phase Peptide Synthesis, A Practical Approach,” W. C. Chan and P. D. White, Eds., Oxford Press, 2000 and elsewhere).

### Methodology for Synthesis of Thioester and Selenoester Compounds

The thioester and selenoester generators of the invention as described above find particular use in the generation of thioester and selenoester compounds. The methods for generating thioester and selenoester compounds in accordance with the invention comprise, in general terms, providing a composition comprising an amino acid synthon having an N-terminal group joined to a C-terminal group through an organic backbone comprising one or more carbons, the organic backbone comprising a carbon having a side chain anchored to a support through a nucleophile-stable linker and lacking reactive functional groups, the C-terminal group comprising a thioester or selenoester moiety, the N-terminal group comprising an unprotected or protected N-terminal group, with the proviso that the N-terminal protecting group is removable under non-nucleophilic conditions, and cleaving the linker under non-nucleophilic conditions to generate a thioester or selenoester compound free of the support. In preferred embodiments, the freed thioester or selenoester compounds are fully or substantially unprotected and are soluble in aqueous solutions.

The above support-bound composition is carried out in generally the same manner as described above for the preparation or generation of thioester and selenoester generators. Cleavage of the linker to form the freed thioester or selenoester may be carried out under various conditions according to the nature of the linker used and the orthogonality of protecting groups present in the composition with respect to the linker. The linker may comprise PAL, XAL, PAB, PAM, SCAL, RINK, WANG, Sieber amides, and other linker systems as described above.

Where an N-terminal protecting group is present, cleavage of the linker may be carried out under conditions orthogonal to removal of the N-terminal protecting group, as well as orthogonal to any protecting groups for side chain groups associated with the amino acid synthon, such that the freed thioester or selenoester compound is fully protected. Such orthogonal conditions may comprise, for example, linker cleavage under acid conditions where the N-terminal protecting group is nucleophile labile. Linker cleavage may alternatively involve non-orthogonal conditions that also result in removal of the N-terminal protecting group and/or one or more amino acid side chain protecting groups that may be present on the organic backbone, such that the freed thioester or selenoester compound is partially protected or unprotected. Selection of various protecting groups and orthogonality of removal of protecting groups with respect to linker cleavage may be made based on desired synthetic schemes and solubility characteristics for the freed thioester or selenoester compounds.

The organic backbone may comprise a residue of an amino acid, peptide, polypeptide, or like moiety comprising alpha, beta, and/or gamma amino acids, and may comprise one or more amino acid side groups which may be protected or unprotected depending upon side group functionality and desired use, as described above. The C-terminal and N-terminal groups may comprise protected or unprotected amino acids, and the N-terminal group may be capable of chemical ligation to form an amide bond or other bond by various ligation techniques, including native chemical ligation and extended native chemical ligation as also described above. In this regard, the N-terminal group in many embodiments may comprise an amino acid with a protected or unprotected side chain functional group that is capable of participating in a chemical ligation reaction, such as thiol or selenol or other group containing a sulfur or selenium atom. The side chain functional group may be associated with a backbone carbon of an N-terminal amino acid or, in the case of extended chemical ligation, be associated with the alpha amine of an N-terminal amino acid.

The methods of generating thioester and selenoester compounds may comprise, more specifically, providing a composition of the formula:

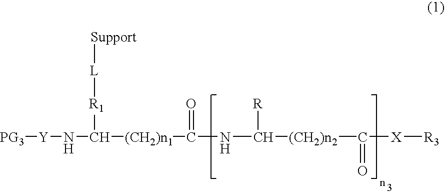

wherein PG₃, Y, R₁, L, Support, R, R₃, X, n₁, n₂, and n₃ are as described above for the structure (1).

Providing the above composition and cleaving of the linker may be carried out as described above, and the groups PG₃, Y, R₁, L, R, R₃ X, n₁, n₂, n₃, and the Support are the same as related above in the description of the thioester and selenoester generators and related methodologies. The thioester or selenoester compound thus freed from the support may comprise the formula:

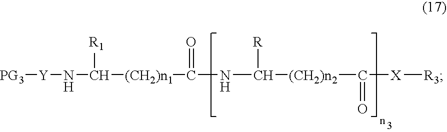

or, where PG₃ is removable under the same conditions used for cleavage of linker L, may comprise the formula:

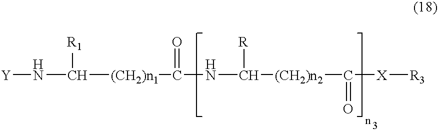

where Y, R₁, R, R₃, X, n₁, n₂, and n₃ are as described above for structure (1).

The invention also provides methods for generating sterically hindered thioester and selenoester compounds, comprising: providing a composition comprising an amino acid synthon having an N-terminal group joined to a C-terminal group through an organic backbone comprising one or more carbons, the organic backbone comprising a carbon having a side chain anchored to a support through a nucleophile-stable linker and lacking reactive functional groups, the N-terminal group comprising an unprotected or protected N-terminal group, the C-terminal group comprising a sterically hindered thioester or selenoester moiety; and cleaving the linker under non-nucleophilic conditions to generate a sterically hindered thioester or selenoester compound free of the support.

The sterically hindered thioester or selenoester compounds freed from the support may be soluble in aqueous solution, and may be protected, partially protected or unprotected as described above. The organic backbone may be associated with a target molecule and may comprise an amino acid, peptide, or polypeptide with one or more side chains bearing protected or unprotected functional groups, and the C-terminal and N-terminal groups may themselves comprise protected or unprotected amino acid groups as also described above. The N-terminal group may be capable of chemical ligation, and may comprise an amino acid with a protected or unprotected side chain functionality capable of participating in native chemical ligation, extended chemical ligation, or other ligation technique to form an amide bond.

The sterically hindered thioester or selenoester compounds may, in certain embodiments, comprise the formula:

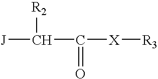

wherein J comprises a residue of the organic backbone; R₂ comprises any side chain group; X is sulfur or selenium; and R₃ is any thioester or selenoester compatible group; and wherein one or more of R₂ and R₃ is a group that sterically hinders the thioester or selenoester moiety —C(O)—X—. More specifically, one or more of R₂ and R₃ may comprise a branching group having the formula:

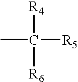

wherein R₄, R₅, and R₆ each individually are hydrogen or a linear, branched, substituted and unsubstituted alkyl, aryl, heteroaryl, and benzyl groups, with the proviso that two or more of R₄, R₅, and R₆ are linear, branched, substituted and unsubstituted alkyl, aryl, heteroaryl, and benzyl groups. The groups X and R₂-R₆ are the same as described above.

The methods for producing sterically hindered thioester or selenoester compounds may more specifically comprise: providing a composition of the formula:

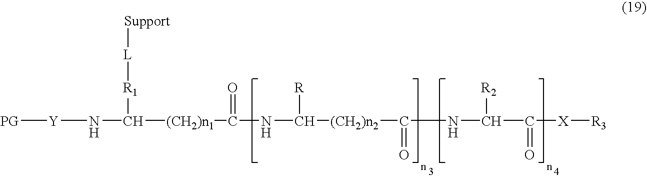

wherein PG, Y, R₁, L, R, R₂, R₃, X, n₁, n₂, n₃, n₄, and the Support are the same as described above for the structure (2); and cleaving the linker L under non-nucleophilic conditions to generate a sterically hindered thioester or selenoester compound free of the support. The sterically hindered thioester or selenoester compound thus freed from the support may have the formula:

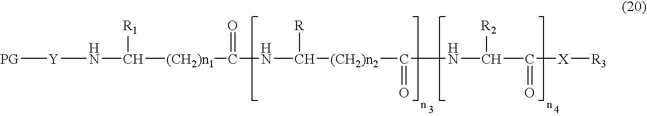

where PG is removable under conditions orthogonal to cleavage of linker L or, where PG is removable under the same conditions used for cleavage of linker L, may comprise the formula:

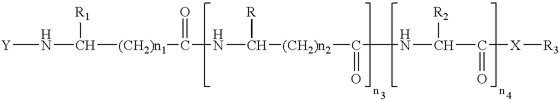

where the groups PG, Y, R₁, L, R, R₂, R₃, X, n₁, n₂, n₃, and n₄ are as provided above.

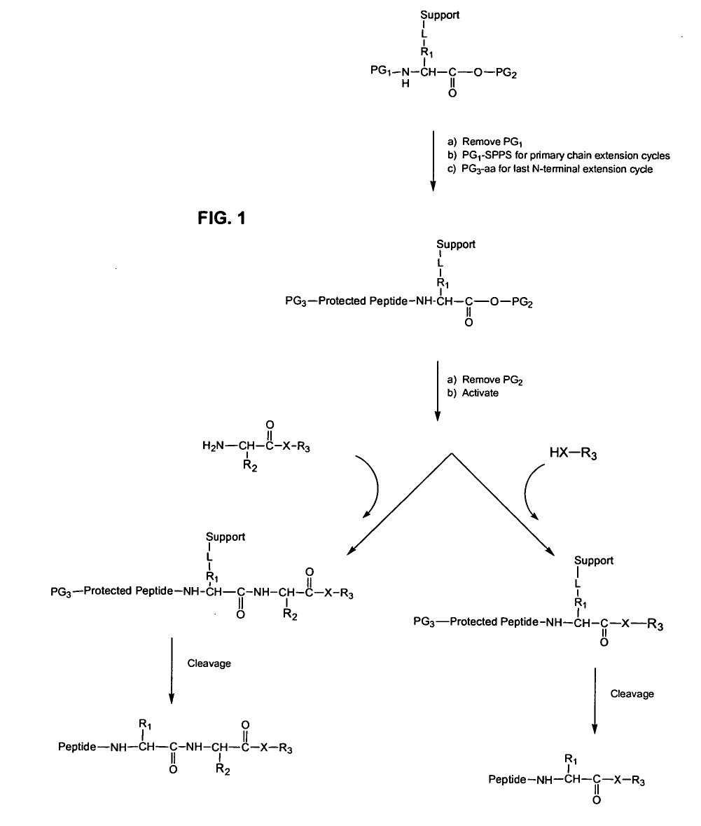

As described above, the invention can be used in nucleophile-based synthesis schemes. The invention finds particular use in the nucleophile-based synthesis of polyamide thioester and selenoester generators, and more particularly, peptide thioester and selenoester generators, and their associated intermediates and products. For instance, the O-alpha-carboxyl group of a side-chain anchored amino acid or peptide is protected with a protecting group that is orthogonal to the nucleophile-labile group used in the SPPS chain assembly chemistry. With Fmoc-SPPS, for example, an allyl, ODmab, or photolytic group may be employed for protecting the C-terminal carboxylate. After SPPS chain-assembly of a selected polyamide is performed from the alpha-amino end of the anchored compound, the alpha-carboxyl group of the anchored compound is deprotected and activated. Then, a preformed amino acid or peptide -thioester or selenoester derivative is acylated with the activated alpha-carboxyl group to provide C-terminal thioester or selenoester functionality usable for subsequent reactions once cleaved from the support, such as, for example, use of the product in chemical ligation reactions. Cleavage of the linker results in the generation of the target thioesters or selenoester product. This embodiment of the invention will be more fully understood by reference to the reaction schemes shown in FIG. 1 through FIG. 3 with respect to preferred compositions and methods for SPPS.

Referring first to FIG. 1, there is an overview of the generation of thioester and selenoester peptides in accordance with the invention. In the reaction scheme of FIG. 1, an amino acid synthon is provided that includes (i) an N-terminal amine protected with a nucleophile labile protecting group PG₁, (ii) a C-terminal carboxyl protected with a carboxyl protecting group PG₂ that is removable under conditions orthogonal to PG₁, and (iii) a side chain R₁ covalently joined to a nucleophile-stable linker L that is cleavable under conditions orthogonal to the carboxyl protecting group PG₂. The amino acid synthon is bound or coupled to a support via linker L. As shown in FIG. 1, the amino acid synthon is a single amino acid, with R₁ corresponding to an amino acid side group capable of binding to linker L. Thus, the amino acid synthon serves as a side chain-anchored thioester- or selenoester-generating precursor usable in SPPS.

As also shown in FIG. 1, for chain extension, the amino acid synthon is extended by a series of addition (deprotection/coupling) cycles that involve adding a Nα-PG₁ protected amino acid or peptide components stepwise in the N- to C-terminal direction. The incoming amino acid or peptides used for chain extension will also have the appropriate side-chain protecting groups present that are stable to nucleophiles and conditions employed for removal of the carboxyl protecting group PG₂. Once chain assembly has been accomplished, a pendant amino acid or peptide is coupled that bears a nucleophile-stable protecting group PG₃, followed by selective removal of PG₂. Removal of PG₂ generates a free carboxylate on the C-terminal end of the elongated peptide. The free carboxylate is then activated to form the carboxyester and reacted with a preformed thioester or selenoester, or a thiol- or selenol-bearing compound. As shown, a preformed thioester or selenoester amino acid is depicted, along with a thiol or selenol reagent, where R₂ (side chain), X (sulfur or selenium), and R₃ (group compatible with thioesters or selenoesters) are as described above. Following conversion of the peptide to the thioester or selenoester, the peptide can be further modified while still bound to the support, or cleaved to release the desired thioester or selenoester peptide. As also shown, the released peptide is deprotected. However, partially protected or even fully protected peptides can be made by employing side chain and/or N-terminal protecting groups stable to the cleavage conditions.

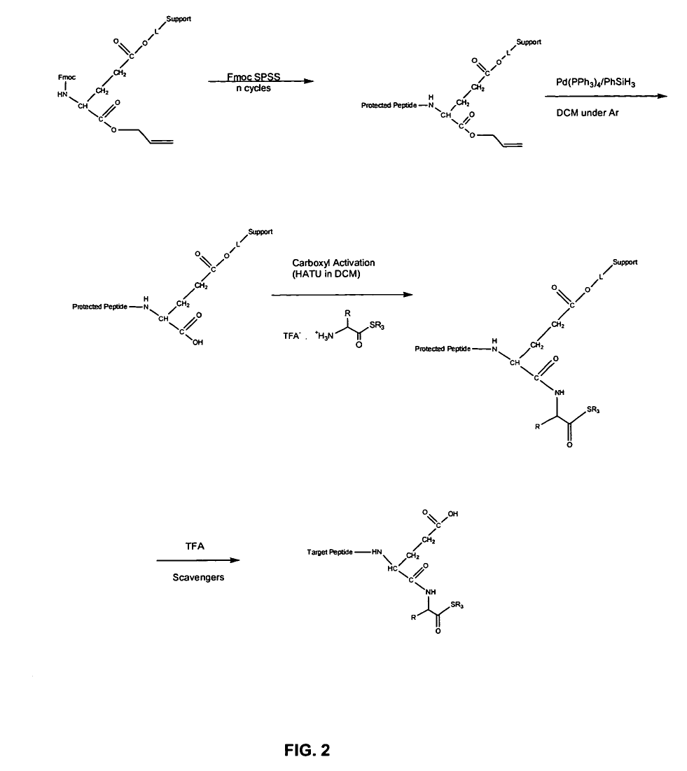

Referring now to FIG. 2, there is schematically shown a specific example of employing a glutamic acid side-chain system for generating a target peptide thioester via Fmoc-SPPS. An Fmoc group protects the Nα-amine, and an allyl group protects the Oα-carboxyl of the amino acid. n cycles of Fmoc SPPS are carried out as described above, so that a protected peptide is “grown” or otherwise formed in the N- to C-terminal direction from the Nα-amine of the amino acid joined to the linker to provide a protected peptide joined or anchored to the linker and support via the glutamate side chain.

The Oα carboxyl allyl protecting group is then removed from the anchored protected peptide under H₂/palladium catalyst conditions, shown in FIG. 2 as Pd(Ph₃)₄/PhSiH₃ in dichloromethane (DCM). Following removal of the allyl protecting group, the Oα carboxyl is activated using N-[(dimethylamino)-1 H-1, 2, 3-triazol [4, 5-b] pyridiylmethylene]-N-methylmethanaminium hexafluorophosphate N-oxide (HATU). The anchored, protected peptide with the activated Oα carboxyl is then reacted with the TFA salt of a preformed amino acid thioester to produce a target peptide thioester anchored to the linker and support. In this example, the backbone side chains of the peptide are protected with acid-labile protecting groups. Thus, TFA as shown in FIG. 2 is used to cleave the linker and release the target peptide thioester from the support and remove the acid-labile side chain protecting groups from the target peptide thioester.

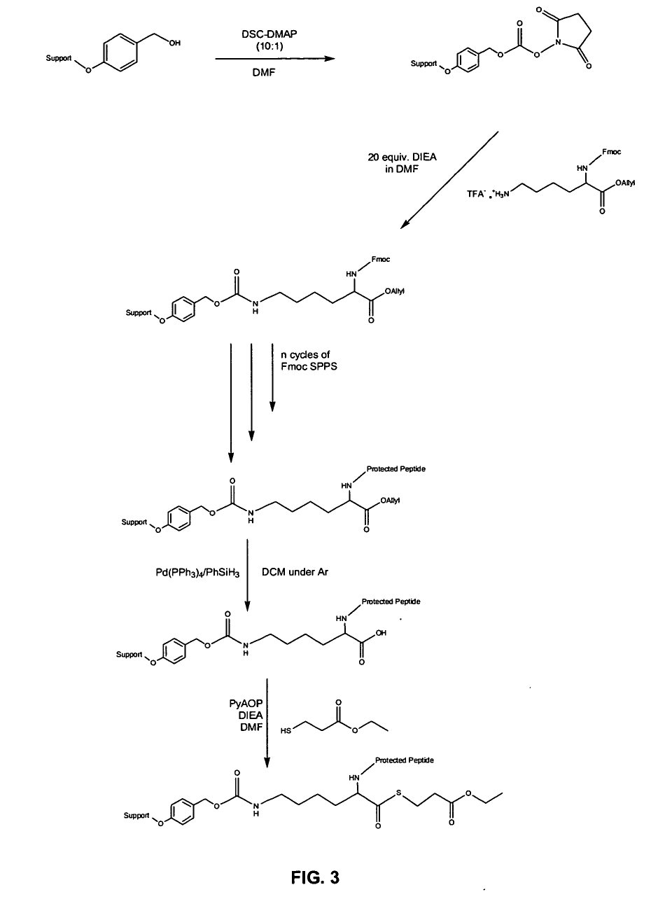

FIG. 3 is a reaction scheme that shows a specific example illustrating the anchoring of an initial amino acid to a support prior to formation of a target peptide thioester. In this example, a lysine amino acid side-chain system is depicted for generating a target peptide thioester via Fmoc-SPPS. In FIG. 3, a support bound WANG linker is treated with N,N′-disuccinimidyl carbonate (DSC)/4-dimethylaminopyridine (DMAP) in N,N-dimethylformamide (DMF) to activate the linker for coupling. The activated linker is then treated with the TFA salt of Nα-Fmoc-Oα-allyl lysine, in N,N-diisopropylethylamine (DIEA)/DMF, to form a linker with a urethane group made with the lysine side chain ε-amino group. The lysine residue thus anchored by its side chain provides an initial basis for Fmoc-based SPPS synthesis, which is carried out to generate a peptide by stepwise growth in the N- to —C-terminal direction from the Nα amine as described above. Once the desired peptide is formed with n cycles of Fmoc-SPPS, the Nα amine is protected with a nucleophile-stable protecting group (not shown in FIG. 3) and the Oα carbonyl is deprotected using H₂/Pd (Pd(Ph₃)₄/PhSiH₃). The free Oα carbonyl is activated using 7-azabenzotroazol-1-1yloxtris (pyrrolidino) phosphonium hexafluorophosphate (PyAOP) in DIEA/DMF, and is reacted with 3-mercapto-propionic acid ethyl ester to produce an anchored target peptide thioester. The target peptide thioester can then be cleaved from the support by treatment with TFA cocktails to yield the free target peptide thioester.

In the reaction scheme of FIG. 3, the Oα thioester is formed on the same amino acid that is anchored to the resin by directly reacting the activated Oα carboxyl of the anchored amino acid with a thiol. This is also shown in FIG. 1. In the reaction scheme of FIG. 2 described above, the ultimate formation of the thioester involves reaction of the Oα carboxyl of the anchored amino acid with an amino acid or peptide thioester.

As can be appreciated, the methods and compositions of the invention as described above, and exemplified in the Examples that follow have wide applicability in organic synthesis for the generation of thioesters and selenoesters. The subject compounds are particularly useful in peptide and polypeptide synthesis techniques that employ thioester and/or selenoester-mediated chemical ligation. Given the broad range of use, the subject thioester and selenoester generators and compounds also may be provided in kits and the like. The invention also allows for the production of activated thioesters and selenoesters from precursors that are prepared under strong nucleophilic conditions or non-nucleophilic synthesis schemes, or a combination of both. Thus, the invention has a wide range of uses and applications.

All publications and patent applications mentioned in this specification are herein incorporated by reference to the same extent as if each individual publication or patent application was specifically and individually indicated to be incorporated by reference.

Having now generally described the invention, the same will be more readily understood through reference to the following examples, which are provided by way of illustration, and are not intended to be limiting of the present invention, unless specified.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used but some experimental errors and deviations should be accounted for. Unless indicated otherwise, parts are parts by weight, molecular weight is weight average molecular weight, temperature is in degrees Centigrade, and pressure is at or near atmospheric.

The following experimental examples provide a detailed description of the Fmoc-based solid-phase synthesis of glutamine and lysine side-chain anchored thioester generators and peptide thioester compounds. Those skilled in the art will recognized that the same or similar procedures described below may be used to synthesize numerous types of thioester and thioester generating compounds. The selenium based- chemistry associated with selenoester formation is well known in the art and, where appropriate, may be substituted. Table 1 provides a list or glossary of abbreviations used in the following experimental examples.

TABLE 1

| | |
|---|---|
| Acm | acetamidomethyl |
| Alloc | allyoxycarbonyl |
| BOP | benzotnazol-1 -yloxytris (dimethylamino) phosphonium |
| | hexafluorophosphate |
| Br, Cl Z | Br, Cl Benzylcarbamate |
| DCM | dichloromethane |
| DDE | 4,4-dimethyl-2,6-dioxocycloex 1-ylidene |
| DIPCDI | N,N-diisopropylcarbodiimide |
| DIEA | N,N-diisopropylethylamine |
| DMAP | 4-dimethylaminopyridine |
| DMF | N,N-dimethylformamide |
| DMSO | dimethylsulfoxide |
| EtOH | ethanol |
| Fmoc | 9-fluorenylmethoxycarbonyl |
| FM | 9-Fluorenylmethyl |
| HATU | (N-[(dimethylamino)-1H-1,2,3-triazol[4,5- |
| | b]pyridiylmethylene]-N-methylmethanaminium |
| | hexafluorophosphate N-oxide). |
| HBTU | N-[(1 -H-benzotriazol-1-yl)(dimethylamine)methylene]-N- |
| | methylmethanaminium hexafluorophosphate N-oxide |
| | previously named 0-(benzotriazol-1-yl)-1,1,3,3- |
| | tetramethyluronium hexafluorophosphate |
| HF | hydrofluoric acid |
| HMP resin | 4-hydroxymethylphenoxy resin; palkoxybenzyl alcohol |
| | resin; or Wang resin |
| HOAt | 1-hydroxy-7-azabenzotriazole |
| HOBt | 1-hydroxybenzotriazole |
| IP10 | Interferon-gamma inducible protein 10 kDa |
| Mbh | dimethoxybenzhydryl |
| MBHA resin | 4-methylbenzhydrylamine resin |
| Meb | p-MethylBenzyl |
| MMA | N-methylmercaptoacetamide |
| Mmt | p-Methoxytriityl |
| Mob | p-MethoxyBenzyl |
| Msc | 2-Methylsulfoethylcarbamate |
| Msz | 4-Methylsulfinylbenzylcarbamate |
| Mtr | 4-methoxy-2,3,6-trimethylbenzene sulfonyl |
| NMM | N-methylmorpholine |
| NMP | N-methylprrolidone,N-methyl-2-pyrrolidone |
| Nsc | 4-nitrophenylethylsulfonyl-ethyloxycarbonyl |
| OPfp | pentafluorophenyl ester |
| OtBu | tert-butyl ester |
| PAC | peptide acid linker |
| PAL | peptide amide linker |
| Pbf | 2,2,4,6,7-pentamethyldihydrobenzofuran-5-sulfonyl |
| PEG-PS | polyethylene glycol-polystyrene |
| Picolyl | methyl-pyridyl |
| Pmc | 2,2,4,6,8-pentamethylchroman-6-sulfonyl |
| PyAOP | 7-azabenzotroazol-1-1yloxtris (pyrrolidino) phosphonium |
| | hexafluorophosphate |
| S-tBu | tert-butyl-thio |
| Tacam | Trimethylacetamidomethyl |
| tBoc | tert-butyloxycarbonyl |
| TBTU | 0-(benzotriazol-1-yl)-1,1,3,3-tetramethyl uronium |
| | tetrafluoroborate |
| tBu | tert-butyl |
| TFA | trifluoroacetic acid |
| Tis | Trisisopropylsilane |
| Tmob | 2,4,6-trimehoxybenzyl |
| TMOF | trimethylorthoformate |
| Troc | 2,2,2Trichloroethylcarbamate |
| Trt | triphenylmethyl |

### Example 1

### Solid Phase Peptide Synthesis

Peptides were synthesized in a stepwise manner on an ABI433 peptide synthesizer by SPPS using HBTU/DIEA/DMF coupling protocols at 0.1 mmol equivalent resin scale. For each coupling cycle, 1 mmol N^{α}-Fmoc-amino acid, 4 mmol DIEA and 1 mmol equivalents of HBTU were used. The concentration of the activated HBTU-activated Fmoc amino acids were 0.5 M in DMF, and the couple time was 10 min. Fmoc deprotections were carried out with two treatments using a 30% piperidine in DMF solution for 2 min and then 18 min.

### Example 2

### Preparation of Nα-Fmoc-Gln(γCONH-Rink Resin)-Oα-Allyl Via MSNT/DCM Mediated Acylation

Rink-resin (0.34 g; 0.87 mmol/g; equiv 0.296 mmol) was swollen in DCM for 5 min. After drainage, the resin was acylated with a solution containing Nα-Fmoc-Glu(OH)-Oα-Allyl (409 mg; 1 mmol), 1-(mesitylene-2-sulfonyl)-3-nitro-1H-1,2,4-triazole (MSNT, 297mg; 1 mmol), N-methylimidazole (0.75 mmol; 75 μL) in anhydrous DCM (2 mL) for 35 min at room temperature. After drainage and washing with DCM, the above acylation procedure was repeated for 1 h. The resin washed with DCM, DMF, and DCM, and dried in vacuo for 3 days.

### Example 3

### Synthesis of Peptide GRFN#1(1-33)[Gln34(γCONH-Rink Resin)-Oα-Allyl]

The Nα-Fmoc-Gln(γCONH-Rink Resin)-Oα-Allyl resin of Example 2 was used to synthesize a glutamine side-chain anchored peptide (GRFN#1) having the amino acid sequence and resin attachment VPLSR TVRCT CISIS NQPVN PRSLE KLEII PASQ(γCONH-Rink Resin)-Oα-Allyl) as described above and with the following side-chain and N-terminal protection strategy: Arginine(Pbf), Asparagine(Trt), Cysteine(Acm), Glutamic acid(OtBu), Glutamine(Trt), Lysine(Nε-Boc), Serine(OtBu), Threonine(OtBu), and Nα-terminal Boc protection (i.e., valine introduce by coupling with Boc-Val-OH).

### Example 4

### Synthesis of 3-(2-Amino-3-phenyl-propionylsulfanyl)-propionic Acid Methyl Ester TFA Salt (Phenylalanine-αCOS-propionic Acid Methyl Ester.TFA Salt)

The TFA salt of a preformed phenylalanine thioester propionic acid methyl ester (Phe35α-COS—CH₂CH₂COOMe) was prepared as follows. To a stirred solution of Boc-phenylalanine (2.65 g, 10 mmol) and HBTU (19.9 mL of a 0.5M solution in DMF, 9.95 mmol) in 10 mL DMF was added DIEA (1.28, 13.4 mmol). The solution was stirred for 1 min and 3-mercapto-propionic acid methyl ester (1.19 mL, 9.9 mmol) in 5 mL DMF added in 2 min and stirred overnight. The reaction was then concentrated in vacuo with co-evaporation with toluene (3×50 mL), taken up in ethyl acetate (100 mL). The organic layer was then washed two times with 0.25M KHSO₄, three times with 10% NaHCO₃, five times with brine, and then water. The organic layer was then collected, dried over Na₂SO₄ for 20 min, and concentrated in vacuo. The residue was analyzed by RP-HPLC (Vydac C₁₈, 0-80% buffer B in 40 min) and did not require any further purification. The ammonium salt was formed by dissolution in 50% TFA in DCM for 30 min followed by repeated concentration in vacuo with DCM. The TFA.ammnonium salt of phenylalanine-αCOS-propionic acid methyl ester was stable at 4° C. for at least 4 weeks. ES/MS: 268 m/z Da. Yield 74%.

### Example 5

### Allyl Deprotection of GRFN#1 (1-33)[Gln34(γ-Rink resin)-Oα-Allyl]

0.025 mmol of GRFN#1(1-33)[Gln34(γ-Rink resin)-Oα-Allyl], as prepared in Example 3, was swollen in dry DCM for 10 min. The C-terminal allyl ester was removed by two treatments with Pd(PPh₃)₄ (10 mg; tetrakis(triphenylphosphine)palladium(0)) DCM in presence of phenylsilane (100 μL; 1.05 mmol) under argon at 25° C. for 30 min. The GRFN#1(1-33)[Gln34(γ-Rink resin)-αOH] resin was then was washed with DCM, DMF, DMF/MeOH, and DCM, and dried in vacuo for 3 h.

### Example 6

### Synthesis GRFN#1(1-34)-Phe35α-COS—CH₂CH₂COOMe

0.025 mmol of GRFN#1(1-33)[Gln34(γ-Rink resin)-αOH], as prepared in Example 5, was swollen in anhydrous DCM (1 mL) for 10 min and then drained. 3-(2-Amino-3-phenyl-propionylsulfanyl)-propionic acid methyl ester TFA salt (0.5 mmol; phenylalanine-S-propionic acid methyl esterTFA salt) as prepared in Example 4 was suspended in DCM (1 mL) and DIEA (1.5 mmol) added. The solution was vortexed at room temperature for 2 min and added to the resin. Solid HATU (0.5 mmol) was then added directly to the resin-mixture, mixed and stirred occasionally for 30 min. The resin was then drained, and washed with DCM, DMF, and DCM, and then dried in vacuo for 1 h. The peptide-resin was then deprotection and released by treatment with a TFA/TIS/H₂O (95:2.5:2.5) solution at room temperature for 1 h. The volatiles were then removed with a stream of nitrogen over 10 min and product extracted with 50% acetonitrile/water. The resin was filtered off and the aqueous solution containing the desired peptide thioester free of the resin was lyophilized. ES/MS: 4364 Da (exp). Calc. 4364.16 Da (Avg.). Yield 30%.

### Example 7

### Preparation of Nα-Fmoc-Gln(γCONH-Rink Resin)-Oα-Allyl Via HBTU/DMF Mediated Acylation

Fmoc-protected Rink-resin (0.34 g; 0.87 mmol/g; equiv 0.296 mmol) was swollen in DCM for 5 min. The Fmoc group was removed with two 30 min treatments with 50% (v/v) piperidine/DMF, and washed thoroughly with DMF (50 mL). After drainage, the resin was acylated with a solution containing Nα-Fmoc-Glu(OH)—Oα-Allyl (409 mg; 1 mmol), N-[(1-H-benzotriazol-1-yl)(dimethylamine)methylene]-N-methylmethanaminium hexafluorophosphate N-oxide (HBTU, 379 mg, 1 mmol) in anhydrous DMF (2 mL) for 1 h at room temperature. After drainage and washing with DMF, the above acylation procedure was repeated for 1 h. The resin washed with DCM, DMF, and DCM, and dried in vacuo overnight.

### Example 8

### Synthesis of GRFN#2(1-26)-Gln27(γCONH-Rink Resin)-Oα-Allyl

The Nα-Fmoc-Gln(γCONH-Rink Resin)-Oα-Allyl resin as prepared in Example 7 was used to synthesize a glutamine side-chain anchored peptide (GRFN#2) having the amino acid sequence and resin attachment CPLQL HVDKA VSGLR SLTTL LRALG AQ(γCONH-Rink Resin)-Oα-Allyl) as described below and with the following side-chain and N-terminal protection strategy: Aspartic acid(OtBu), Arginine(Pbf), Cysteine(Acm), Glutamic acid(OtBu), Glutamine(Trt), Histidine(Trt), Lysine(Nε-Boc), Serine(OtBu), Threonine(OtBu), and Nα-terminal Boc protection (i.e., cysteine is introduced by coupling with Boc-Cys(Acm)-OH). For each coupling cycle, 1 mmol Nα-Fmoc-amino acid, 4 mmol DIEA and 1 mmol equivalents of HBTU were used. The concentration of the activated HBTU-activated Fmoc amino acids were 0.5 M in DMF, and the couple time was 10 min. Fmoc deprotections were carried out with two treatments using a 30% (v/v) piperidine in DMF solution for 2 min and then 18 min.

### Example 9

### Allyl Deprotection of GRFN#2(1-26)[Gln27(γ-Rink resin)-Oα-Allyl]

0.1 mmol of GRFN#2(1-26)[Gln27(γ-Rink resin)-Oα-Allyl] as prepared in Example 8 was swollen in dry DCM for 10 min. The C-terminal allyl ester was removed by two treatments with Pd(PPh₃)₄ (25 mg, tetrakis(triphenylphosphine)palladium(0)) DCM in the presence of phenylsilane (100 μL; 1.05 mmol) with continuous argon purging at 25° C. for 30 min. The GRFN#2(1-26)[Gln27(γ-Rink resin)-αCOOH] resin was then was washed with degassed DCM, DMF, DMF/MeOH, and DCM, and dried in vacuo for 3 h. Importantly, the DCM solution was purged with argon for 20 min before used and the Pd(PPh₃)₄ exposure to air and light was minimize. It is recommended, that weighing and storage of Pd(PPh₃)₄ also be done under argon and the reactant kept at −20° C. for storage and in darkness after weighing and during reaction.

### Example 10

### Synthesis GRFN#2(1-27)-Lys28α-COS—CH(CH₃)₂

0.1 mmol of GRFN#2(1-26)[Gln27(γ-Rink resin)-αCOOH] as prepared in Example 9 was swollen in anhydrous DCM (1 mL) for 10 min under argon and then drained to prepare this resin. A preformed sterically hindered lysine thioester (2-Amino-6-tert-butoxycarbonylamino-hexanethioic acid S-isopropyl ester TFA salt (1 mmol; TFA⁻.⁺NH₃—CH[(CH₂)₄—NH—Boc]COS—CH(CH₃)₂)) was suspended in DCM (2 mL) and DIEA (3 mmol) was added. The solution was vortexed at room temperature until dissolved (˜2 min but no longer than 5 min) and was added to the resin. Solid HATU (1 mmol) was then immediately added directly to the resin-mixture, mixed and stirred occasionally for 30 min. This procedure was repeated. The resin was then drained, and washed with DCM, DMF, and DCM, and then dried in vacuo for 1 h. The peptide-resin was then deprotection and released by treatment with a TFA/TIS/H₂O (95:2.5:2.5) solution at room temperature for 1 h. The volatiles were then removed with a stream of nitrogen over 10 min, precipitated twice with diethyl ether, and separated by centrifugation, and the product extracted with 50% acetonitrile/water. The resin was filtered off and the aqueous solution was lyophilized. Calc. mass: 3118.82 Da (average).

### Example 11

### Preparation of Nα-Fmoc-Lys(εNH-Trityl Resin)-αO-Allyl

2-Chlorotrityl chloride resin (0.833 g; 1.2 mmol/g; equiv 1 mmol) was swollen is DCM for 5 min. The resin was treated two times with Nα-(9-fluorenylmethyoxycarbonyl)-lysine O-allyl ester, trifluoroacetate salt (20 mmol) and 40 mmol DIEA in DMF (10 mL). The resin was drained and washed with DMF between treatments. After the reaction, the resin washed with DCM, DMF, and DCM, and dried in vacuo overnight. Loading was determined spectrometrically by the quantitation of dibenzofulvene-piperdine by-product after Fmoc cleavage with 50% piperidine/DMF from a standard curve.

### Example 12

### Synthesis of GRFN#3(1-27)[Lys28(εNH-Trityl Resin)-αO-Allyl]

The Nα-Fmoc-Lys(εNH-Trityl Resin)-αO-Allyl resin as prepared in Example 11 was used to synthesize a lysine side-chain anchored peptide (GRFN#3) having the amino acid sequence and resin attachment CPLQL HVDKA VSGLR SLTTL LRALG AQK(εNH-Trityl resin)-Oα-Allyl as described below and with the following side-chain and N-terminal protection strategy: Aspartic acid(OtBu), Arginine(Pbf), Cysteine(Acm), Glutamic acid(OtBu), Glutamine(Trt), Histidine(Trt), Lysine(Nε-Boc), Serine(OtBu), Threonine(OtBu), and Nα-terminal Boc protection (i.e., cysteine is introduce by coupling with Boc-Cys(Acm)-OH). For each coupling cycle, 1 mmol Nα-Fmoc-amino acid, 4 mmol DIEA and 1 mmol equivalents of HBTU were used. The concentration of the activated HBTU-activated Fmoc amino acids were 0.5 M in DMF, and the couple time was 10 min. Fmoc deprotections were carried out with two treatments using a 30% (v/v) piperidine in DMF solution for 2 min and then 18 min.

### Example 13

### Allyl Deprotection of GRFN#3(1-27)[Lys28(εNH-Trityl resin)-αO-Allyl]

0.1 mmol of GRFN#3(1-27)[Lys28(εNH-Trityl resin)-αO-Allyl] resin, as prepared in Example 13, was swollen in dry DCM for 1 h. The C-terminal allyl ester was removed by two treatments with Pd(PPh₃)₄ (25 mg; tetrakis(triphenylphosphine)palladium(0)) DCM in the presence of phenylsilane (100 μL; 1.05 mmol) with continuous argon purging at 25° C. for 30 min. The G1713(89-116)[Lys116(εNH-Trityl resin)-αCOOH] resin was then was washed with degassed DCM, DMF, DMF/MeOH, and DCM, and dried in vacuo for 3 h.

### Example 14

### Synthesis GRFN#3(1-27)[Lys28-αCOS—CH₂CH₂COOEt]

0.1 mmol of GRFN#3(1-27)[Lys(28εNH-Trityl Resin)-αCOOH] resin was swollen in anhydrous DCM (1 mL) for 10 min. The thiol reagent HS—CH₂CH₂-COOEt (10 mmol) in DMF (2 mL) and DIEA (3 mmol) were added, and the reaction mixed thoroughly. Solid PyAOP (or DIC) (1 mmol) was then immediately added directly to the resin-mixture, mixed thoroughly and left for 1 h. This coupling procedure was repeated. The resin was then drained, and washed with DCM, DMF, and DCM, and then dried in vacuo for 1 h. This procedure was repeated. The resin was then drained, and washed with DCM, DMF, and DCM, and then dried in vacuo for 1 h. The peptide-resin was then deprotected and released by treatment with a TFA/TIS/H₂O (95:2.5:2.5) solution at room temperature for 1 h. The volatiles were then removed with a stream of nitrogen over 10 min, precipitated twice with diethyl ether and separated by centrifugation, and the product was extracted with 50% acetonitrile/water. The resin was filtered off and the aqueous solution containing the desired peptide-thioester free of the resin was lyophilized.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A thioester or selenoester generator comprising an amino acid synthon having an N-terminal group joined to a C-terminal group through an organic backbone comprising one or more carbons, said organic backbone comprising a carbon having a side chain anchored to a support through a nucleophile-stable linker and lacking reactive functional groups, said N-terminal group comprising an unprotected or protected N-terminal group, with the proviso that the protecting group of said protected N-terminal group is removable under non-nucleophilic conditions, and said C-terminal group comprising a moiety selected from the group consisting of a thioester or selenoester.

2. A thioester or selenoester generator having the formula: 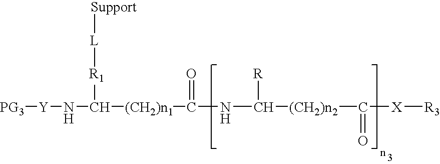
wherein PG₃ is a nucleophile-stable protecting group that may be present or absent; Y is a target molecule of interest that may be present or absent and is lacking reactive functional groups; Support is a solid phase, matrix, or surface; L is a nucleophile-stable linker; R₁ is a divalent radical lacking reactive functional groups; R is hydrogen or an organic side-chain lacking reactive functional groups; n₁ and n₂ each are from 0 to 2; n₃ is from 0 to 20; X is sulfur or selenium; and R₃ is any group compatible with thioesters or selenoesters.

3. A sterically hindered thioester or selenoester generator comprising an amino acid synthon having an N-terminal group joined to a C-terminal group through an organic backbone comprising one or more carbons, said organic backbone comprising a carbon having a side chain anchored to a support through a nucleophile-stable linker and lacking reactive functional groups, said the N-terminal group comprises a unprotected or protected N-terminal group, and the C-terminal group comprises a moiety selected from the group consisting of a sterically hindered thioester or selenoester.

4. A sterically hindered thioester or selenoester generator having the formula: 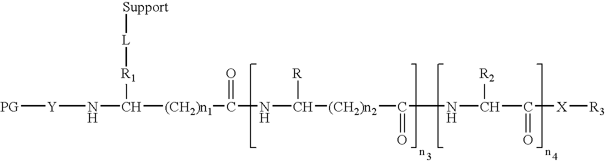
wherein PG is a protecting group that may be present or absent; Y is a target molecule of interest that may be present or absent and is lacking reactive functional groups; Support is a solid phase, matrix, or surface; L is a nucleophile-stable linker; R, is a divalent radical lacking reactive functional groups; each R individually is any side chain group and may be the same or different, each R₂ comprises any side chain group, and R and R₂ are lacking reactive functional groups; n₁ and n₂ each individually is 0, 1 or 2; n₃ is 0 to 20; n₄ is 0 or 1; X is sulfur or selenium; and R₃ is any thio selenoester compatible group; and wherein one or both of R₂ and R₃ is a group that sterically hinders the thioester or selenoester moiety —C(O)—X—.

5. A method of production for a thioester or selenoester generator, said method comprising:
(a) providing a composition comprising an amino acid synthon having an N-terminal group joined to a C-terminal group through an organic backbone comprising one or more carbons, said organic backbone comprising a carbon having a side chain anchored to a support through a nucleophile-stable linker and lacking reactive functional groups, said N-terminal group comprising an unprotected or protected N-terminal group, with the proviso that said N-terminal protecting group is removable under non-nucleophilic conditions, and said C-terminal group comprising a free carboxyl; and
(b) converting said free carboxyl of the product step (a) to a thioester or selenoester.

6. A method of production for a thioester or selenoester generator, said method comprising:
(a) providing a composition having the formula: 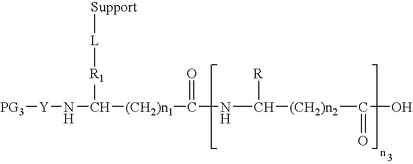
wherein PG₃ is a nucleophile-stable protecting group that may be present or absent; Y is a target molecule of interest that may be present or absent and is lacking reactive functional groups; Support is a solid phase, matrix, or surface; L is a nucleophile-stable linker; R₁ is a divalent radical lacking reactive functional groups; R is hydrogen or an organic side-chain lacking reactive functional groups; n₁ and n₂ each are from 0 to 2; and n₃ is from 0 to 20; and
(b) converting the free carboxyl of step (a) to a thioester or selenoester to form a thioester or selenoester generator having the formula: 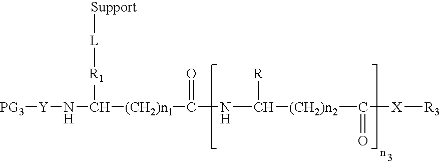
wherein X is sulfur or selenium; and R₃ is any group compatible with thioesters or selenoesters.

7. A method of production for a sterically hindered thioester or selenoester generator, said method comprising:
(a) providing a composition comprising an amino acid synthon having an N-terminal group joined to a C-terminal group through an organic backbone comprising one or more carbons, said organic backbone comprising a carbon having a side chain anchored to a support through a nucleophile-stable linker and lacking reactive functional groups, said N-terminal group comprising an unprotected or protected N-terminal group, and said C-terminal group comprising a free carboxyl; and
(b) converting said free carboxyl of the product step (a) to a sterically hindered thioester or selenoester.

8. A method of production for a sterically hindered thioester or selenoester generator, said method comprising:
(a) providing a composition having the formula: 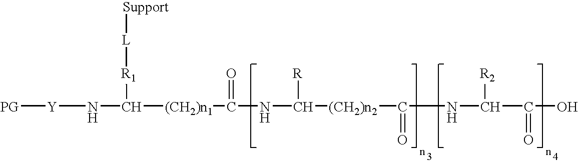
wherein PG is a protecting group that may be present or absent; Y is a target molecule of interest that may be present or absent and is lacking reactive functional groups; L is a nucleophile-stable linker; Support is a solid phase, matrix, or surface; R₁ is a divalent radical lacking reactive functional groups; R and R₂ each individually are any side chain group that may be the same or different and are lacking reactive functional groups, and wherein R₂ is any group compatible with thioesters or selenoesters; n₁ and n₂ each individually is 0, 1 or 2; n₃ is 0 to 20; and n₄ is 0 or 1; and
(b) converting the free carboxyl of step (a) to a sterically hindered thioester or selenoester having the formula: 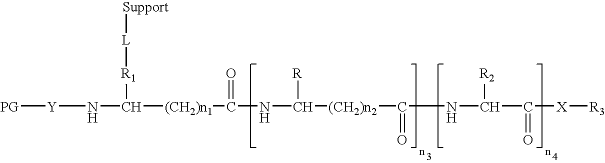
wherein X is sulfur or selenium; and R₃ is any group compatible with thioesters or selenoesters; and wherein one or both of R₂ and R₃ is a group that sterically hinders the thioester or selenoester moiety —C(O)—X—.

9. A method of production for a thioester and selenoester compound, said method comprising:
(a) providing a thioester or selenoester generator comprising an amino acid synthon having an N-terminal group joined to a C-terminal group through an organic backbone comprising one or more carbons, said organic backbone comprising a carbon having a side chain anchored to a support through a nucleophile-stable linker and lacking reactive functional groups, said N-terminal group comprising an unprotected or protected N-terminal group, with the proviso that the N-terminal protecting group is removable under non-nucleophilic conditions, and said C-terminal group comprising a moiety selected from the group consisting of a thioester or selenoester; and
(b) cleaving said linker under non-nucleophilic conditions to generate a thioester or selenoester compound free of said support.

10. A method of producing a thioester and selenoester compound, said method comprising:
(a) providing a thioester or selenoester generator having the formula: 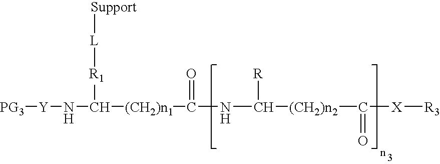
wherein PG₃ is a nucleophile-stable protecting group that may be present or absent; Y is a target molecule of interest that may be present or absent and is lacking reactive functional groups; L is a nucleophile-stable linker; Support is a solid phase, matrix, or surface; R₁ is a divalent radical lacking reactive functional groups; R is hydrogen or an organic side-chain lacking reactive functional groups; n₁ and n₂ each are from 0 to 2; n₃ is from 0 to 20; X is sulfur or selenium; and R₃ is any group compatible with thioesters or selenoesters; and
(b) cleaving linker L under non-nucleophilic conditions to generate a thioester or selenoester compound free of said support, said thioester or selenoester compound having a formula selected from the group consisting of:

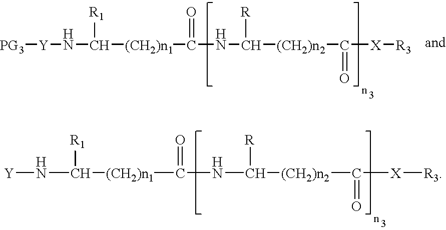

11. A method of producing a sterically hindered thioester or selenoester compound, said method comprising:
(a) providing a thioester or selenoester generator comprising an amino acid synthon having an N-terminal group joined to a C-terminal group through an organic backbone comprising one or more carbons, said organic backbone comprising a carbon having a side chain anchored to a support through a nucleophile-stable linker and is lacking reactive functional groups, said N-terminal group comprising an unprotected or protected N-terminal group, and said C-terminal group comprising a moiety selected from the group consisting of a sterically hindered thioester or selenoester; and
(b) cleaving said linker under non-nucleophilic conditions so as to generate a sterically hindered thioester or selenoester compound free of said support.

12. A method of producing a sterically hindered thioester or selenoester compound, said method comprising:
(a) providing a thioester or selenoester generator having the formula: 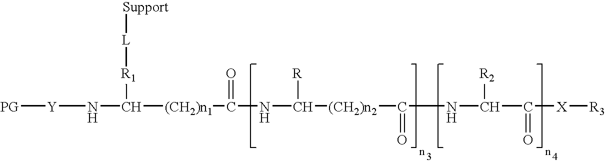
wherein PG is a protecting group that may be present or absent; Y is a target molecule of interest that may be present or absent and is lacking reactive functional groups; L is a nucleophile-stable linker; Support is a solid phase, matrix, or surface; R₁ is a divalent radical lacking reactive functional groups; each R individually is any side chain group and may be the same or different, each R₂ comprises any side chain group, and R and R₂ are lacking reactive functional groups; n₁ and n₂ each individually is 0, 1 or 2; n₃ is 0 to 20; n₄ is 0 or 1; X is sulfur or selenium; and R₃ is any thioester compatible group; and wherein one or more of R₂ and R₃ is a group that sterically hinders the thioester or selenoester moiety —C(O)—X—; and
(b) cleaving linker L under non-nucleophilic conditions to generate a sterically hindered thioester or selenoester compound free of said support, said sterically hindered thioester or selenoester compound having a formula selected from the group consisting of:

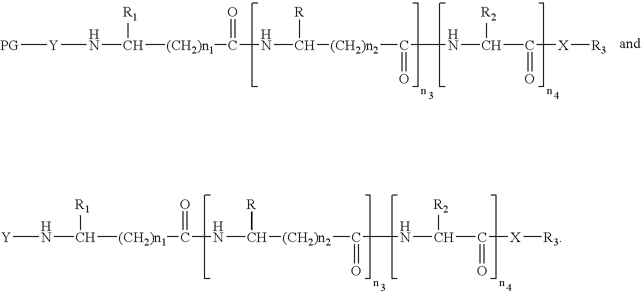

13. A method of nucleophile-based production of a thioester or selenoester generator, said method comprising:
(a) providing a composition comprising an amino acid synthon having an N-terminal group joined to a C-terminal group through an organic backbone comprising one or more carbons, said N-terminal group comprising a reactive functional group protected with a nucleophile-labile protecting group, said C-terminal group comprising a carboxyl protected with a carboxyl protecting group removable under conditions orthogonal to said nucleophile-labile protecting group and said organic backbone lacking reactive functional groups and comprising a carbon having a side chain anchored to a support through a nucleophile-stable linker cleavable under conditions orthogonal to the carboxyl protecting group;
(b) removing said nucleophile-labile protecting group from said composition of step (a) under nucleophile conditions and forming an N-terminal group comprising a first reactive functional group;
(c) coupling to the product of step (b), a compound forming a covalent bond with said first reactive functional group to form an elongated product, where the compound is selected from a group consisting of: (i) an unprotected compound comprising a single reactive moiety that forms said covalent bond with said first reactive functional group; (ii) a protected compound comprising a single reactive moiety that forms said covalent bond with said first reactive functional group, and an amine protected with a nucleophile-stable amino protecting group removable under conditions orthogonal to removal of said carboxyl protecting group; and (iii) a protected compound comprising a single reactive moiety that forms said covalent bond with said first reactive functional group and one or more additional reactive functional groups protected with a protecting group removable under conditions orthogonal to removal of said carboxyl protecting group;
(d) removing from the product of step (c), said carboxyl protecting group to generate a free carboxyl group; and
(e) converting said free carboxyl group to produce a thioester or selenoester.

14. A method of nucleophile-based production of a thioester or selenoester generator, said method comprising:
(a) providing a thioester or selenoester generator having the formula: 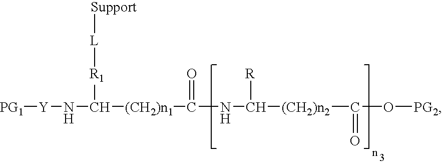
wherein PG₁ is a nucleophile-labile protecting group that may be present or absent; Y is a target molecule of interest that may be present or absent and is lacking reactive functional groups; Support is chosen from a solid phase, matrix, or surface; L is a nucleophile-stabile linker; R₁ is a divalent radical lacking reactive functional groups; R is hydrogen or any organic side-chain lacking reactive functional groups; n₁ and n₂ each are from 0 to 2, and n₃ is from 0 to 20; and PG₂ is any protecting group that is removable under conditions orthogonal to removal of PG₁ and cleavage of L;
(b) removing said nucleophile-labile protecting group from the composition of step (a) to generate a composition having the formula: 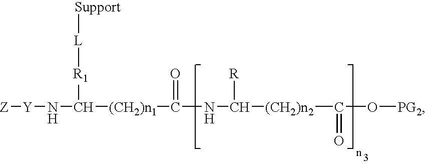
wherein Z comprises a reactive functional group of interest;
(c) coupling said reactive functional group of the composition of step (b) to a compound of interest and forming an elongated product having the formula: 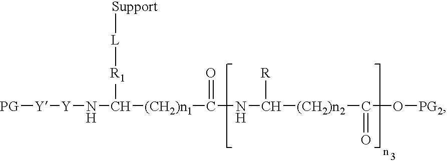
wherein Y′ is a compound of interest lacking reactive functional groups; and PG may be present or absent, with the proviso that when present, PG is a nucleophile-stable amino protecting group removable under conditions orthogonal to PG₂ and Y′ comprises an N-terminal amino group that is protected by PG;
(d) removing said carboxyl protecting group from the product of step (c) to generate a free carboxyl group having the formula: 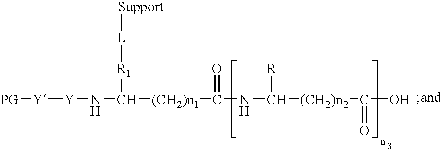
and
(e) converting the product of step (d) to a thioester or selenoester of the formula: 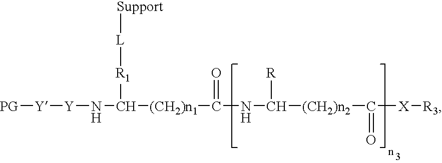
wherein X is sulfur or selenium; and R₃ is any group compatible with thioesters or selenoesters.

15. A method of nucleophile-based production of a sterically hindered thioester or selenoester generator, said method comprising:
(a) providing a composition comprising an amino acid synthon having an N-terminal group joined to a C-terminal group through an organic backbone comprising one or more carbons, said N-terminal group comprising a reactive functional group protected with a nucleophile-labile protecting group, said C-terminal group comprising a carboxyl protected with a carboxyl protecting group removable under conditions orthogonal to said nucleophile-labile protecting group and said organic backbone lacking reactive functional groups and comprising a carbon having a side chain anchored to a support through a nucleophile-stable linker cleavable under conditions orthogonal to the carboxyl protecting group;
(b) removing said nucleophile-labile protecting group from said composition of step (a) under nucleophile conditions and forming an N-terminal group comprising a first reactive functional group;
(c) coupling to the product of step (b), a compound forming a covalent bond with said first reactive functional group to form an elongated product, where the compound is selected from a group consisting of: (i) an unprotected compound comprising a single reactive moiety that forms said covalent bond with said first reactive functional group; (ii) a protected compound comprising a single reactive moiety that forms said covalent bond with said first reactive functional group, and an amine protected with a nucleophile-stable amino protecting group removable under conditions orthogonal to removal of said carboxyl protecting group; and (iii) a protected compound comprising a single reactive moiety that forms said covalent bond with said first reactive functional group and one or more additional reactive functional groups protected with a protecting group removable under conditions orthogonal to removal of said carboxyl protecting group;
(d) removing from the product of step (c), said carboxyl protecting group to generate a free carboxyl group; and
(e) converting said free carboxyl group to produce a thioester or selenoester, with the proviso that the converting the product of step (d) formed from the elongated product of step (c)(iii) comprises generating a sterically hindered thioester or selenoester.

16. A method of nucleophile-based production of a thioester or selenoester generator, said method comprising:
(a) providing a thioester or selenoester generator having the formula: 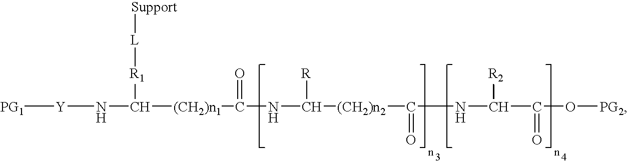
wherein PG₁ is a nucleophile-labile protecting group that may be present or absent; Y is a target molecule of interest that may be present or absent and is lacking reactive functional groups; Support is chosen from a solid phase, matrix, or surface; L is a nucleophile-stabile linker; R₁ is a divalent radical lacking reactive functional groups; R and R₂, each individually, are hydrogen or any organic side-chain lacking reactive functional groups; n₁ and n₂, each individually, are from 0 to 2, n₃ is from 0 to 20, n₄ is 0 or 1; and PG₂ is any protecting group that is removable under conditions orthogonal to removal of PG₁ and cleavage of L;
(b) removing said nucleophile-labile protecting group from the composition of step (a) to generate a composition having the formula: 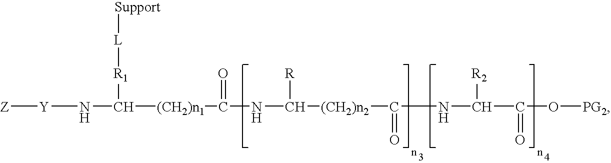
wherein Z comprises a reactive functional group of interest;
(c) coupling said reactive functional group of the composition of step (b) to a compound of interest and forming an elongated product having the formula: 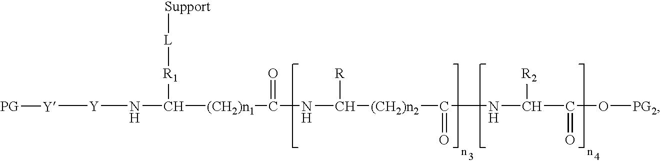
wherein Y′ is a compound of interest lacking reactive functional groups; and PG may be present or absent, with the proviso that, if present, PG is a nucleophile-stable amino protecting group removable under conditions orthogonal to PG₂;
(d) removing said carboxyl protecting group from the product of step (c) to generate a free carboxyl group having the formula: 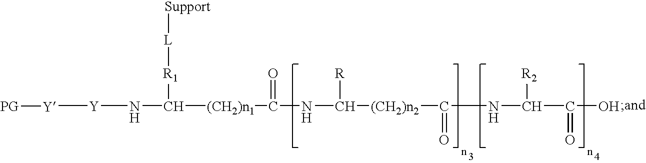
and
(e) converting the product of step (d) to a thioester or selenoester of the formula: 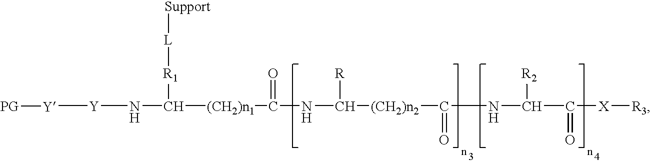
wherein X is sulfur or selenium; R₂ is one or more of any group that sterically hinders said thioester or selenoester; and R₃ is any group compatible with thioesters or selenoesters; and wherein one or both or R₂ and R₃ is a group that sterically hinders said thioester or selenoester.
